(19) United States
(12) Patent Application Publication
(10) Pub. No.: US 2021/0361837 A1
(43) Pub. Date: Nov. 25, 2021

(54) METHODS AND APPARATUS TO IMPROVE BREAST MILK PRODUCTION

(71) Applicant: NIRVANTAGE INC., Sunnyvale, CA (US)

(72) Inventor: Sheila BIJOOR, Sunnyvale, CA (US)

(21) Appl. No.: 17/392,741

(22) Filed: Aug. 3, 2021

Related U.S. Application Data
(63) Continuation of application No. 16/208,166, filed on Dec. 3, 2018.
Application No. 16/208,166 is a continuation of application No. 15/061,926, filed on Mar. 4, 2016, now abandoned.
(60) Provisional application No. 62/128,852, filed on Mar. 5, 2015.

(51) Int. Cl. A61M 1/06

(52) U.S. Cl. CPC A61M 1/064; A61M 2205/3365; A61M 1/066

(57) ABSTRACT

A breast pump having an areola stimulation assembly is provided. The breast pump may include a breast cup. The breast pump may also include a vacuum component that connects to the breast cup. Additionally, the breast pump may include an areola stimulation assembly, contained within the breast pump, that comprises an upper plate; a lower plate; and a mechanism that moves the lower plate with respect to the upper plate.

### CROSS-REFERENCE TO RELATED APPLICATIONS

This application is a continuation-in-part of U.S. patent application Ser. No. 16/208,166 filed Dec. 3, 2018, which is a continuation of U.S. patent application Ser. No. 15/061,926 filed on Mar. 4, 2016 that claims the benefit of U.S. Provisional Patent Application Ser. No. 62/128,852 entitled “Methods and Apparatus to Improve Breast Milk Production,” filed Mar. 5, 2015, the disclosures of which is hereby incorporated by reference in its entireties.

### BACKGROUND OF THE INVENTION

Breast pumps are commonly used amongst lactating women. Breast pumps may be used for convenience when a nursing woman is away from her child. They may also be used to extract milk from the breast to treat engorgement, provide milk for a child that is hospitalized, or provide milk when breastfeeding is uncomfortable or impossible such as when a baby cannot latch to the breast

Despite their wide usage, however, breast pumps are generally associated with discomfort, frustration, isolation, and inefficiency. Pumping often takes place under stressful conditions, such as during work breaks and in bathroom stalls. As a result, women often experience difficulty or delay in achieving the milk ejection reflex—the process by which milk is released from milk glands—while pumping as compared to breastfeeding a baby. In general, breast pumping is considered to be less effective and efficient than a baby at stimulating production and extraction of milk from the breast. Most breast pumps are unwieldy and indiscreet, making it difficult to multi-task or be mobile while pumping.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### BRIEF SUMMARY OF THE INVENTION

Various aspects of the invention involve improved breast pumping systems, devices, and techniques, including various embodiments of pumping apparatus including breast pumps and/or milk expression assemblies incorporating various types of areolar stimulation assemblies. In at least one embodiment, a breast pump assembly and associated components can include a breast cup which is placed over the nipple and which encompasses some or all of the nipple, the areola and/or portions of the surrounding fatty tissues of the breast.

In various embodiments, a breast pump assembly may include a vacuum component or other negative pressure inducing source that connects to the breast cup. In other alternative embodiments, a breast pump assembly may include a pressurized component or other positive pressure inducing source that connects to the breast cup. In still other embodiments, a source of electrical or mechanical energy may be provided to the breast cup. Alternatively, various combinations thereof may be provided to the breast cup.

In various embodiments, a breast pump assembly may include one or more breast cup assemblies. A breast cup assembly may optionally include connection features between a plurality of breast cups.

In various embodiments, a breast cup assembly can include an areolar stimulation assembly, which may be contained partially or fully within the breast cup, partially or fully within the breast pump, or partially or fully within an accessory component. In at least one exemplary embodiment, an areolar stimulation assembly can comprise an upper plate, a lower plate, and a mechanism that moves at least a portion of the lower plate relative to the upper plate.

In another aspect of the invention, a breast pump is provided. The breast pump may include an outer cup for accommodating at least a distal portion of a breast; a first element positioned within the cup, the first element connected to an inner surface of the outer cup and including a generally first rigid base and a first surface for engaging with a first skin surface of the breast; a second element positioned within the cup, the second element moveable with respect to the first element and including a second generally rigid base and a second surface for engaging with a second skin surface of the breast; and the first surface of the first element including a curved surface shaped and configured to emulate a baby's upper palate, and the second surface of the second element including a surface shaped and configured to emulate a baby's tongue, the first and second surfaces being spaced apart and facing towards each other, wherein, when the breast pump is placed over the distal portion of the breast, at least a portion of the breast tissue is positioned between the first and second elements, and movement of the second element relative to the first element causes the first and second surfaces to engage the first and second skin surfaces.

In some examples, the movement of the second element periodically cycles between a first position and a second position, a first spacing between the first and second surfaces when the second element is in the first position being less than a second spacing between the first and second surfaces when the second element is in the second position, the first and second surfaces compressing at least a portion of the breast tissue therebetween when the second element is in the first position. In some examples, the first and second elements are located at diametrically opposed positions within the outer cup. In some examples, the breast pump may include a moveable linkage connecting the second element to the outer cup, the moveable linkage including a motor and a controller element, the controller element capable of controlling a movement speed of the second element. In some examples, the breast pump may include a moveable linkage connecting the second element to the outer cup, the moveable linkage including a motor and a controller element, the controller element capable of controlling a movement path of the second element. In some examples, the breast pump may include a flexible liner positioned within the breast cup. In additional examples, the liner may comprise a heating element. In some examples, the connection between the first element and the inner surface of the outer cup is adjustable by a user. In some examples, the first surface comprises a generally soft, flexible material. In some examples, at least a portion of the outer cup is conically shaped.

In a further aspect of the invention, a breast pump is provided. The breast cup may include a breast cup for accommodating at least a portion of a user's breast; a vacuum source connected to the breast cup, the vacuum source operatively connected to a controller, the controller programmed to provide a first baseline vacuum to the breast cup and a second maximum vacuum to the breast cup, at least a portion of the second maximum vacuum being at a vacuum pressure greater than the first baseline vacuum; and a mechanical stimulator positioned within the breast cup, the mechanical stimulator including at least a first element and a second element, the first element moveable with respect to the second element.

In some examples, the controller is programmed to operate said vacuum source in a manner that results in a vacuum waveform that cycles between the first baseline vacuum and the second maximum vacuum. In some examples, the first baseline vacuum creates a retention force sufficient to maintain the breast cup on the breast in a desired position. In some examples, the user can selectively adjust a frequency of the cycles between the first baseline vacuum and the second maximum vacuum. In some examples, wherein the user can selectively adjust a pressure of the second maximum vacuum. In some examples, the user can selectively adjust a pressure of the first baseline vacuum.

In an additional aspect of the invention, a breast pump is provided. The breast pump may include a breast cup for accommodating at least a portion of a breast; a first element and a second element at least partially located within the cup, at least one of the first and second elements moveable with respect to the cup, the first and second elements each including a surface for engaging at least a portion of the breast; and an actuating mechanism for engaging at least a distal portion of the breast, the actuating mechanism securing the distal portion of the breast within the breast cup.

In some examples, the actuating mechanism comprises a mechanical capture feature which engages with and draws the portion of the breast into the breast cup. In some examples, the breast pump further comprises a vacuum source connected to the breast cup, wherein the actuating mechanism comprises the vacuum source which provides a first baseline vacuum to draw the portion of the breast into the breast cup. In some examples, the actuating mechanism further elongates the distal portion of the breast within the breast cup.

In another aspect of the invention, a breast pump component is provided. The breast pump component may include a vacuum source connected to a breast cup, the vacuum source operatively connected to a controller, the controller programmed to provide a first baseline vacuum to the breast cup and a second maximum vacuum to the breast cup, at least a portion of the second maximum vacuum being at a vacuum pressure greater than the first baseline vacuum.

### BRIEF DESCRIPTION OF THE SEVERAL VIEWS OF THE DRAWINGS

Novel features of the invention are set forth with particularity in the appended claims. A better understanding of the features and advantages of the invention will be obtained by reference to the following detailed description that sets forth illustrative embodiments, in which the principles of the invention are utilized, and other objects, aspects, features, and advantages of embodiments will become more apparent and may be better understood by referring to the description taken in conjunction with the accompanying drawings, in which:

FIG. 1 shows a block diagram of a breast pump system, in accordance with embodiments of the invention;

FIG. 2 shows a block diagram of mechanical components of the breast pump system of FIG. 1, in accordance with embodiments of the invention;

FIGS. 3A and 3B illustrate a comparison of a vacuum pressure waveform utilized in prior art breast pumps without baseline suction and a vacuum pressure waveform utilized in breast pumps having an established baseline suction, in accordance with embodiments of the invention;

FIG. 4 illustrates a progressive waveform utilized in breast pumps, in accordance with embodiments in the invention;

FIG. 5A illustrates components of a breast pump, in accordance with embodiments of the invention;

FIG. 5B a front view of the breast pump of FIG. 5A, in accordance with embodiments of the invention;

FIGS. 5C and 5D depict additional alternative embodiments of the breast pump of FIG. 5A, in accordance with embodiments of the invention;

FIG. 6A illustrates components of a breast pump having an inflatable bladder, in accordance with embodiments of the invention;

FIG. 6B illustrates components of an additional embodiment of a breast pump, in accordance with embodiments of the invention;

FIG. 7A illustrates components of a breast pump that attaches primarily around an areola, in accordance with embodiments of the invention;

FIG. 7B illustrates components of a breast pump that attaches primary at a distal portion of a breast, in accordance with embodiments of the invention;

FIG. 8 illustrates components of a breast pump having multiple vacuum ports, in accordance with embodiments of the invention;

FIG. 9 illustrates a vacuum source control panel, in accordance with embodiments of the invention;

FIG. 10 provides a block diagram of breast pump structural components, in accordance with embodiments of the invention;

FIGS. 11A and 11B provide an illustration of compression within a stimulation mechanism, in accordance with embodiments of the invention;

FIG. 12A-12B depicts a cross-sectional view of a breast, in accordance with embodiments of the invention;

FIG. 12C-12D depicts a side and front cross-sectional view of a breastfeeding infant and breast, in accordance with embodiments of the invention;

FIG. 13A depicts a view of one embodiment of a stimulating element, in accordance with embodiments of the invention;

FIGS. 13B and 13C depict an exemplary movement profile for a stimulating element, in accordance with embodiments of the invention;

FIGS. 14A through 14C depict another embodiment of an exemplary movement profile for a stimulating element, in accordance with embodiments of the invention;

FIG. 15 depicts a cross-sectional side view of one embodiment of tongue peristaltic action;

FIGS. 16A-16B depicts a side and front view of one embodiment of a breast pump;

FIGS. 17A-17F depicts a side view of one embodiment of the latch and swallow/suck sequence with simplified view of the plates/pads of the breast pump; and

FIGS. 18A-18C depicts one embodiment of a method for breast pump activation.

### DETAILED DESCRIPTION OF THE INVENTION

Disclosed are devices, systems and methods for stimulating the areola, nipple and/or other areas of the breasts and related anatomy of human females and/or other mammals. Various embodiments can be utilized to desirably stimulate the production of milk from human and/or mammalian milk glands, such as to establish or improve breast milk production. Devices, systems, and methods discussed herein may also be used to stimulate various anatomical regions such as the areola and nipple for a variety of purposes, including employment as a sex toy or relaxation aid and/or to promote improved fluid circulation, reduce scarification after breast surgery and/or enhance breast size.

Breast pumps can be utilized to induce or improve milk production, and may also be utilized for collection and storage of a “reserve” milk supply for an infant, which may be used for convenience when a nursing woman is away from her child. Breast pumps may also be used to extract milk from the breast to treat engorgement, provide milk for a child that is hospitalized, or provide milk when breastfeeding is uncomfortable or impossible.

However, breast pumps are generally poorly designed. Aside from various ergonomic factors involving the poor design and usability of the pumping apparatus and milk collection devices, breast pumping often requires extended pumping periods (typically in excess of 30 minutes each pumping session) with numerous pumping sessions spread throughout the day (to accommodate the continued creation of milk and limited storage capacity of the average breast). For many women, pumping sessions often take place under stressful conditions, which leads women to experience difficulty or delay in achieving the milk ejection reflex—the process by which milk is released from milk glands—while pumping as compared to breastfeeding a baby. In general, breast pumping is considered to be significantly less effective and efficient than a baby at stimulating production and extraction of milk from the breast, with many studies calculating that breast pumping produces less than half the amount of milk as a comparable infant breastfeeding. In addition, most breast pumps are large, heavy, unwieldy and indiscreet, further making it difficult for a breast pumping woman to multi-task or be mobile while pumping.

Accordingly, the present disclosure describes devices, systems and methods for improving the production and collection of milk from human and/or mammalian mammary glands, including a variety of apparatus for improving the efficiency and user “experience” of utilizing a milk expression and collection apparatus or “breast pump” and associated systems. In various embodiments, the disclosed systems include a stimulation apparatus for physically manipulating the nipple and/or areola of the user to induce, enhance, and/or improve the expression and/or collection of breast milk in a much more efficient and/or pleasurable manner than with standard, commercially-available vacuum operated breast pumps.

In various disclosed embodiments, breast pumps, milk expression devices, and various related systems are described that can be utilized for a variety of purposes, including (but not limited to) milk extraction, relactation, lactation induction, labor induction, as simulation devices and/or sex toys, and/or to enhance breast size and/or induce repair/replacement of damaged and/or missing tissues.

In general, the human breast is adapted to respond optimally to suckling by a human baby, whose lips tend to move rhythmically as the baby suckles, massaging the breast. This massaging action stimulates the discharge of milk. Merely attempting to suck milk from the nipple is typically much less effective, because the breast and hormonal systems of the mother may not respond well to simple suction absent additional stimuli.

While nursing, a baby may latch on to the nipple of the breast and sucks quickly until milk is flowing into her mouth. While some smaller amount of milk may be immediately available from the breast, it may take a minute or two for milk to start flowing in larger quantities. When the milk is flowing at a faster rate, such as during a milk rejection reflex, the baby's sucking motion may slow down, but when milk flow slows, the baby may begin to suck faster in an attempt to start another milk ejection reflex or “let-down.” Most mothers have an average of four milk ejection reflexes during a typical breastfeeding session.

During breastfeeding, the baby may draw the nipple and some portion of the areola into her mouth using physical manipulation and/or mouth suction. Accordingly, the baby may gently massage and/or cycle the mother's areola and nipple with its tongue and upper palate. This physical manipulation may create a pleasurable sensation for the mother, may aid in squeezing milk ducts, and may promote the release of hormones required for milk ejection.

Hand techniques to stimulate the nipple so as to mimic the motion of the baby's mouth may be employed in the processes of relactation (reinitiating lost milk supply) or lactation induction (establishing a milk supply when none is present, such as when adopting a baby). In addition to physical stimulation, the baby's mouth typically latches to the breast using a gentle, constant suction and may extract milk with periodic sucks.

Current breast pumps primarily utilize a vacuum effect to induce milk production. However, these current breast pumps fail to stimulate the breast and suck milk in a way that generates the same or similar effects as a breastfeeding baby. In general, breast pumps are often associated with discomfort, inactivity, isolation, frustration, inefficiency, and sometimes even pain for the pumped individual. As such, there is a need for an improved breast pump that improves efficiency, improve convenience, provides a pleasurable sensation, provides nipple and/or areola stimulation, and improves wearability and/or usability of the breast pump. In various embodiments, the disclosed breast pumps and associated components can be utilized to (1) decrease the time to milk ejection; (2) increase the output and efficiency of milk extraction; (3) increase the comfort during milk extraction through the nipple stimulation; (4) increase the comfort during pumping with improved waveform(s) that have less push and/or pull on the breast; and/or (5) enable pumping while reclining, sleeping or walking.

While breast pumps can also be used to aid in relactation (restarting milk supply) or lactation induction (establishing a milk supply when none is present, such as when adopting a baby), breast pumps may be suboptimal substitutes for current “best practices,” which can include non-nutritive suckling by a baby, hand manipulation of the breast, and hormone therapy. In many cases, the vacuum effect utilized for milk production by existing breast pumps does not adequately imitate the hand technique or a baby's physical manipulation of the areola and nipple, and thus does not promote hormone release leading to milk letdown.

In addition to improving the experience and efficiency of breast pumping, there also exists an opportunity to devise a breast pump or similar device which stimulates the nipple and/or areola, and which can be used as an adjunct to standard pumping devices and/or simply a device for enhancing sexual arousal and pleasure. Nipple stimulation is a common practice to initiate and enhance sexual arousal. Methods of physical manipulation and nipple stimulation can include sucking, biting, rubbing, and squeezing. A breast pump or other device that effectively, conveniently, and pleasurably stimulates the nipple may be employed as a sex toy. As in breastfeeding, nipple stimulation can promote the release of oxytocin and prolactin. Oxytocin and prolactin are hormones which encourage relaxation and feelings of bonding. Current sex toys that stimulate the nipple primarily utilize vibration and/or pinching. However, these sex toys do not provide the sensation and motion of a baby's mouth during breastfeeding to generate pleasurable responses.

Additionally, there is an opportunity for a breast pump or similar device that stimulates the nipple and/or areola, and which can be used as a device to enhance the size of the breast or portions thereof (i.e., nipple and/or areola). Nipple stimulation and breast massage may be utilized to temporarily or permanently increase the size of the breast and/or portions thereof as a result of sexual arousal, fluid retention, tissue growth, and/or scarification, if desired. In a similar manner, nipple stimulation and/or breast massage may be useful adjuncts for physical therapy of the breasts and related tissues after surgical procedures such as mastectomy, breast reduction and/or augmentation. In various embodiments, nipple and/or areolar stimulation (and/or breast stimulation) can include one or more of the following, including kneading, twirling, stroking, the use of (or simulation of) multiple fingers or palm (i.e., a flat or curved plate), movement with use of a lubricant such as cocoa butter or olive oil, cupping or lifting on the bottom, or pushing together of surfaces and/or the breasts and upward movement (i.e., similar to the way a woman can ‘create cleavage’ by pushing breasts together in an upward motion).

In various additional embodiments, a baseline suction or other self-adhering feature can be incorporated into a breast pump or other milk expression device to desirably maintain the device in a desired position and/or orientation on the breast and/or portions thereof. Self-adhesion of the breast cups can provide a significant contribution to enhanced wearability and usability of the system, such as a hands-free operation and/or pumping while nursing, sleeping, working, exercising, and/or performing household chores. These functionalities are not optimized by devices lacking in a baseline suction or other retention feature so as to ensure milk does not spill out of the device when a lactating woman is reclining. In addition, the large size and/or bulkiness of the collection devices as well as the pumping apparatus and associated power supplies may limit the mobility of the pumping systems and/or the mobility of the person using the pumping systems.

In various embodiments, the stimulation, milk expression and/or suction aspects of breast pumps and associated components, as discussed herein, might include monitoring and/or feedback features, including optional automatic assessment and/or adjustment by a controller in real-time, according to feedback collected in the controller by the apparatus. Such aspects can include the collection and monitoring (and potentially remote control) of milk collection and/or pump performance using a smartphone or other device with computing capability, which may include the use of wireless communications between the breast pump, the mobile computing device and/or the internet.

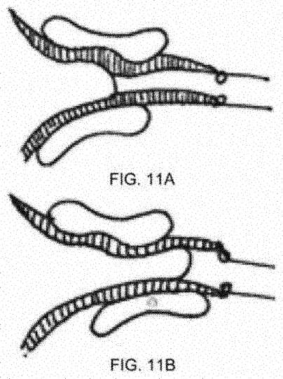

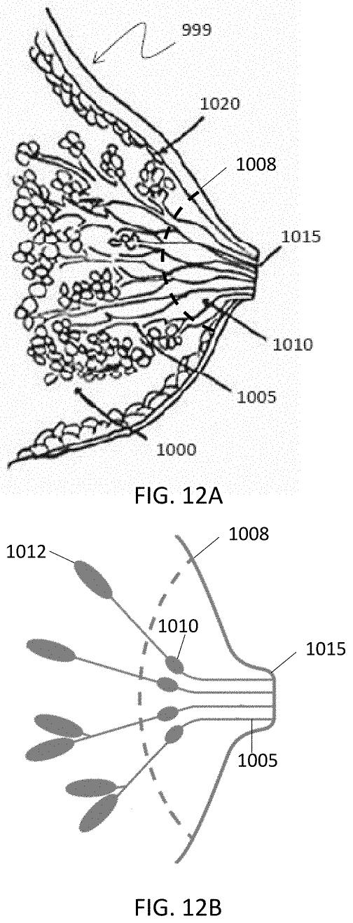

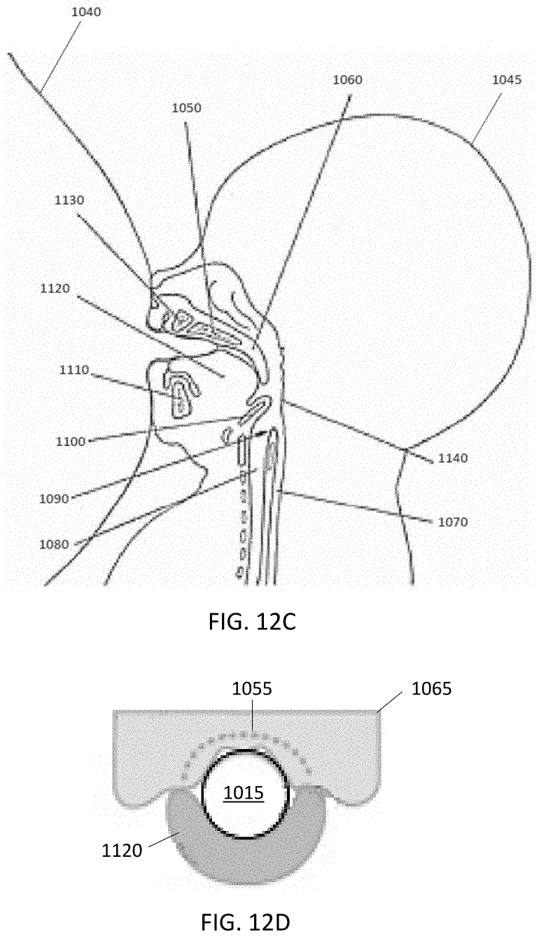

Milk “letdown” is a term applied to the release of milk into the mother's lactiferous ducts (i.e., breast ducts) in response to the baby's suckling. At birth, milk is created within lactocytes or cuboid cells of the breast 999 (see FIG. 12A), which are surrounded by myoepithelial cells (which are themselves contained within alveoli 1000 of the mammary glands) surrounded by lipid or fat cells 1020. Lobules 1012 of the alveoli are drained by the lactiferous ducts 1005, which connect through the milk sinuses 1010 to openings 1015 in the nipple (i.e., nipple pores). Accordingly, the areolar 1008 is also an important part of the breastfeeding process because it is also crucial for continued stimulation of breast milk production. The areolar 1008 is a circular or oval area of skin on the breast 999 that is usually darker in shade that rest of chest skin (not shown). The areolar 1008 comprising a diameter, the diameter includes an average range of 1 to 2 inches (or about 25 mm to 51 mm). Disposed within the areolar 1008 or disposed lower in the areolar 1008 towards the nipple 1015, comprises milk ducts 1005 and the milk sinuses 1010. A correct latch includes the entire nipple 1015 with at least 1 inch (25 mm) of the areolar to initiate the stimulation of the flow of milk. FIG. 12B depicts a simplified cross-sectional side view of the breast 999 and its components for milk production.

Suckling by the baby stimulates the mechanoreceptors in the nipple 1015, which send ascending sensory impulses to the mother's spinal cord, which pass to the hypothalamus. The hypothalamus sends an inhibitory signal to the prolactin inhibitory neurons, which decreases the flow of a prolactin inhibitor within the anterior pituitary gland, resulting in an increase in the flow of prolactin from the anterior pituitary gland. The increased prolactin levels induce the creation of milk within the mammary glands. The hypothalamus also activates oxytocin neurons of the posterior pituitary gland to release the hormone oxytocin into the blood stream. The increased oxytocin stimulates the myoepithelial cells surrounding the outer walls of the alveoli to contract, ejecting milk from the mammary glands into the lactiferous ducts 1005 (e.g., milk ducts). Approximately 30 seconds after the baby begins to suck, milk flows down the lactiferous sinuses into the milk sinuses 1010 or “reservoirs” under the areola 1008, which is then expelled out of the nipple 1015 pores for consumption by the baby.

To breastfeed effectively, a baby needs to coordinate three basic processes: suck, swallow and breathe. FIG. 12C depicts a breast 1040 and cross-sectional image of a breastfeeding baby 1045, with various structures of the baby's mouth and throat, including the hard palate 1050, the soft palate 1060, the esophagus 1070, the trachea 1080, the larynx 1090, the epiglottis 1100, the mandible 1110, the tongue 1120, the alveolar ridge 1130 and the pharyngeal wall 1140. Breastfeeding is primarily accomplished by manipulation of various components of the oral cavity, including the lips, the upper jaw, the lower jaw, the cheeks, tongue, floor of the mouth, gum ridges, hard palate, soft palate and uvula. For example, the lips help locate the nipple, bringing it into the mouth and stabilizing the position of the nipple-areolar complex within the mouth.

FIG. 12D depicts a front view (or anterior view) of the nipple 1015 straddled by the upper jaw 1065 and the tongue 1120. The suckling fossa 1055 is a dent in the center of the upper jaw of an infant. The perimeter of the nipple 1015 is surrounded by the upper jaw 1065 and the tongue 1120 to latch on to the breast for breastfeeding.

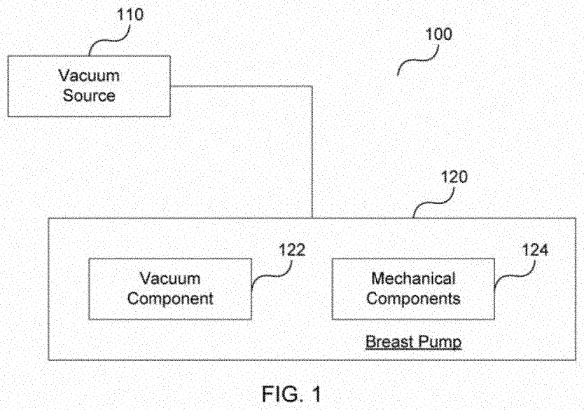

FIG. 1 shows a block diagram of one exemplary embodiment of a breast pumping apparatus incorporating various features of the disclosed invention. In this embodiment, a breast pump system 100 comprises a vacuum or suction source 110 and a breast pump 120, where the breast pump can have both a vacuum component 122 and mechanical components 124. The vacuum component 122 may include tubing that connects to the vacuum source 110. The vacuum component 122 may be used for a variety of purposes, which can include establishing suction to affix the breast pump 100 to the breast, drawing of the nipple and/or areola into a desired portion of the pump, to extract milk from the breast and/or to transfer milk from the pump to a storage location. Depending upon the devices employed and/or the objectives in using the vacuum, the strength and/or frequency of pressure/vacuum that is applied may vary, as discussed below.

In various embodiments, mechanical and/or pneumatic components 124 may be used to stimulate, massage and/or otherwise manipulate the areola, nipple and/or other areas of the breast, which may include the use of mechanical elements alone to induce milk production as well as combinations of mechanical and vacuum/pressure milk inducement. The massaging by mechanical and/or pneumatic components 124 may be performed in a variety of manners, including in various ways that may simulate the massage and/or other manipulation of the nipple, areola and/or breast that is provided by a baby's mouth during nursing. In particular, one or more mechanical components 124 may be used to simulate the movement of a baby's tongue and/or upper palate while nursing, thereby desirably stimulating potions of the breast, and desirably causing increased pleasure and/or milk production for the nursing woman. For example, a light stroking or squeezing of the sides of the nipple during pumping and lactation can potentially enhance the expression of milk, in a manner akin to the suckling of an infant.

In various embodiments, a recording of a baby's cry may be provided for the milking individual to hear, which may provide additional stimulation to promote let-down of milk during pumping.

In various embodiments, a breast pump or cup can optionally be provided that fits over the nipple and/or areola and/or other portions of the breast. The cup may be rigid or elastic, or incorporate both types of material. For example, if the cup needs to open to allow insertion of the breast, then it may have elastic portions enabling it to open. One or more breast cups can encompass some portion of the nipple, areola and/or breast tissues, which in various embodiments can house the various areola/nipple stimulation and/or suction mechanisms. The cup may optionally include a heating element to warm the cup and/or to contribute to comfort and increased/decreased milk let down time. The cup may incorporate materials that enhance comfort for the user, including the use of materials and/or colors that may be pleasing to the user and/or comfortable for extended skin contact. If desired, the inner surface and/or edge of the cup may adhere to the skin, such as for enhanced wearability. Alternatively, the cup may be placed on the nipple and held in place by hand, may be alternately affixed to the breast only when sufficient suction is applied and/or it may be affixed to breast when baseline suction is applied.

In various embodiments, a liner can be provided that attaches and isolates the bladders, plates, rollers, air pipes and/or any electromechanical components from the breast tissue and milk. This liner can be removed for disinfecting and/or switching with replaceable pieces according to user preference, and may be disposable. The lining at the opening of the cup may be made of material to enhance the seal within the cavity.

The breast cup can include moveable elements having one or more surfaces and/or protruding fingers extending from the inner edges of the surfaces and positioned to stroke and/or massage the surface of the nipple, areola and/or breast to stimulate the release of milk from the breast ducts. The fingers or other surface features need not all be of a same length and/or texture, and in at least one embodiment they can be of two or more different lengths and/or shapes to massage different parts of the breast or massage in differing manners on different movement of the elements (i.e., during a forward and “return” stroke). The moveable elements and/or surface features may be flat, curved and/or generally spoon-shaped, with the bowl of the “spoon” forming a surface of the element, and a concave and/or convex surface of the bowl contacting the breast. The surface may include tips or other surface features formed with small bumps to enhance the stimulating effect of the massage, if desired.

The various moveable elements described herein may be disposed in a variety of configurations, including the use of 1, 2, 3, 4, 5, 6, 7, 8 or more moveable elements within a single breast cup, which may be utilized in combination with non-moveable elements, if desired. For example, the moveable elements may include two or more diametrically opposed surfaces that come into contact with the areola. In various embodiments, the upper surface and lower surfaces of the moveable elements may incorporate shapes, configurations and/or materials that may simulate the baby's upper palate in size, shape or material feel. Desirably, the one or both surfaces can comprise elements that enable these surfaces to act upon the areola and nipple.

If desired, the distance between a plurality of surfaces may be adjustable to produce varying levels of compression on the areola and nipple at various places, with the surfaces shaped such that, during motion the nipple would not experience excessive friction and/or chafing, which may be painful.

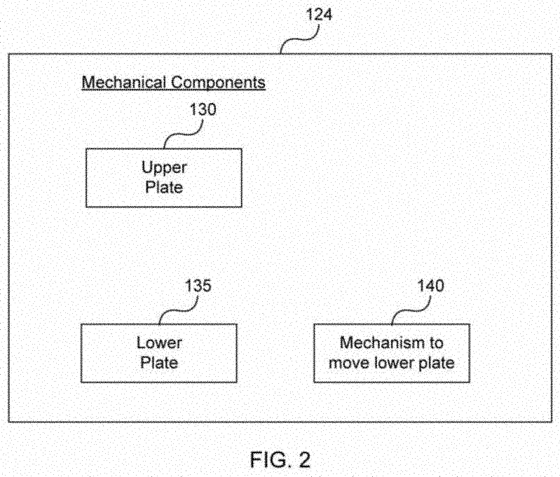

FIG. 2 shows a block diagram of one embodiment of exemplary mechanical components for employment with the breast pump system of FIG. 1, which can include an upper plate 130 that may simulate a baby's upper palate; a lower plate 135 that may simulate a baby's tongue; and a mechanism to move the lower plate 140. In this embodiment, the lower plate 135 is desirably moveable relative to the upper plate, but in other embodiments the upper plate may be moveable relative to the lower plate, and/or both of the plates may be moveable relative to each other and/or relative to other portions of the system. In various additional embodiments, the lower plate may be moveable relative to the upper plate along one or more directions, while the upper plate may be moveable relative to the lower plate along the same or differing directions.

Accordingly, the mechanical components 124 may mimic the hard and soft palates within a baby's mouth during breastfeeding to massage the areola and/or nipple, and/or suck milk from the breast. The mechanical components 124 of the breast pump system 100 may be used to stimulate milk ejection, stimulate let-down reflex, express milk from the lactiferous ducts (i.e., lactiferous sinuses), milk sinuses and/or nipple pores, provide relaxation and/or pleasure including sexual arousal (such as a sex toy), or stimulate nipples for inducing labor, relactation, or lactation.

In one embodiment, the lower plate may be moveable in at least one direction, at least one movement type and at least one frequency. The at least one movement type may comprise opening-closing movement, peristaltic waves, and anterior posterior linear movement, and/or any combination thereof. The at least one direction may comprise a vertical direction, a horizontal direction, an oblique direction and/or any combination thereof. The at least one frequency may comprise at least 1 Hz to 2 Hz. Alternatively, the at least one frequency may be adjustable from 1 Hz or greater, at 0.25 Hz increments.

In various embodiments, a plurality of subsystems may be provided, with each subsystem performing a differing and/or complimentary function, such as a milk collection unit, a nipple and/or areola stimulator, and/or a tracking and monitoring subsystem. Each of these subsystems may be a standalone system that may be used on its own or in combination with one or more of the other subsystems, or may be utilized with independent devices. For example, a milk collection unit may include a vacuum system that extracts milk from the breast into a flange or storage reservoir. The nipple and/or areolar stimulator may compress and/or manipulate various breasts tissues and the tracking and monitoring system may evaluate and monitor milk collection. The system may desirably include an engine or power supply. In various embodiments, the engine may comprise one or more vacuum pumps, a linear, rotary or reciprocating motor, a hydraulic unit, a powered collection unit and/or a positive pressure pump, or various combinations thereof. If desired, the engine may be used to power multiple subsystems. For example, a pump may have a vacuum port for powering a milk collection unit and/or a positive pressure port for powering the nipple and/or areolar stimulation components. The system may optionally include a controller that may control operation and coordinate operation of one or more subsystems, or some or all of the subsystems may include a separate controller.

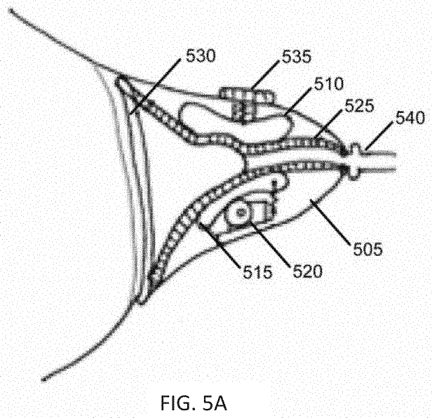

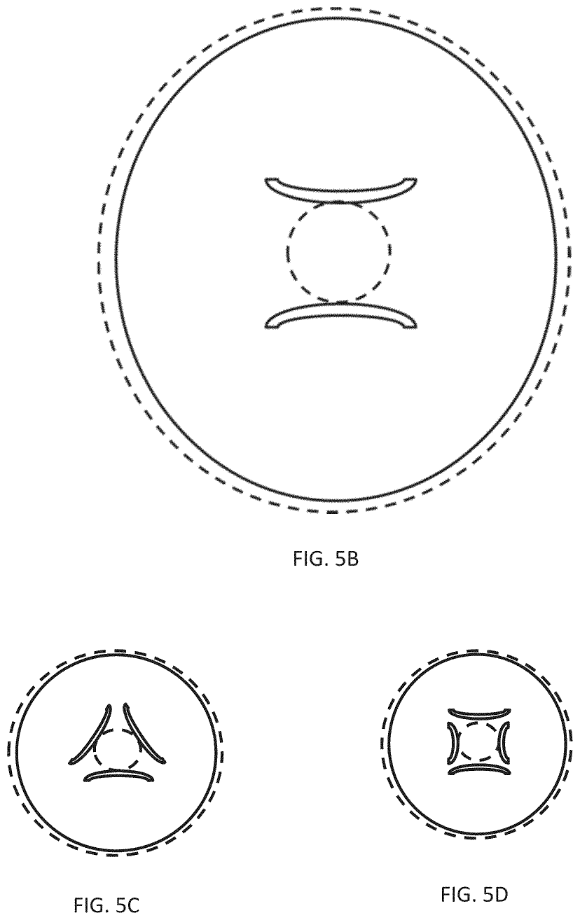

FIGS. 5C and 5D depict additional arrangements for the distribution of mechanical stimulating components that may be utilized with various embodiments of the invention. For example, FIG. 5C depicts three diametrically opposed plates, while FIG. 5D depicts 4 plates, with lateral of smaller dimensions than the upper and lower plates. Combination of rollers or combination of plates or one or. In additional examples, mechanical stimulating components can include one or more rollers, can include one or more plates, or can include a combination of rollers and plates. Additionally, the mechanical stimulating components provided may be equally spaced from one another, may be unequally spaced from one another, or may be a combination of different spacing.

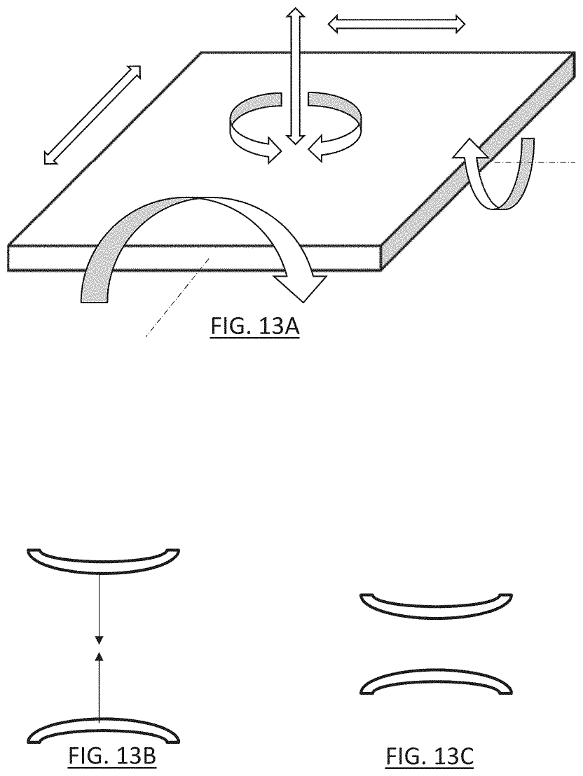

FIG. 13A depicts a schematic view of one mechanical stimulating component or “plate” showing various exemplary motion patterns that may be utilized in conjunction with various embodiments of the invention. The plate may follow the path of one or more of the depicted arrows, or various combinations thereof for more complex motions. In addition, various portions of the plate may experience differing motion than other portions of the plate, if desired. In addition, other plate motions not depicted herein may be utilized. In examples, motions that may be utilized may include horizontal and vertical motion, only vertical motion, circular motion, rolling, pressing, squeezing, or other types of motions. In additional examples, a combination of motions may be used. In some examples, the profile of motion may look like squeezing, for example, where the motion may be a “rolling” or peristaltic pressure wave.

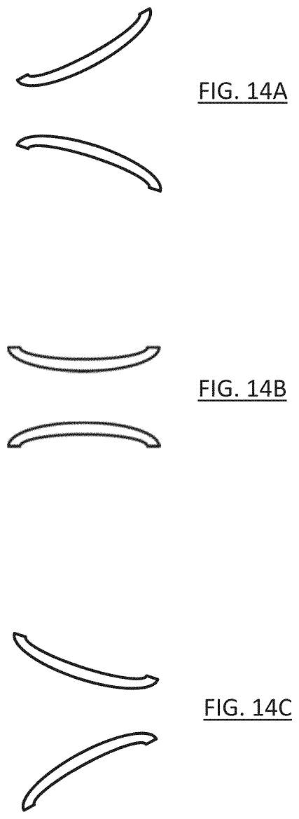

FIGS. 13B and 13C depict one exemplary motion of opposing plates, which may include plate motions towards and/or away from each other to produce a compressing or pinching force therebetween. FIGS. 14A, 14B and 14C depict another exemplary motion of opposing plates, which may include a rocking or “rolling” motion that produces a progressive compressing or “wave” force.

The plates may take many shapes, including the flat plate of FIG. 13A or the gently curved plates with a convex surface facing forward towards an opposing plate of FIG. 13B, with a concave rearward surface. If desired, the exemplary flat plate of FIG. 13A may be utilized as a base plate, which may be a fixed or moveable component. The various plates described herein may comprise a relatively rigid material such as wood, metal or plastic, which may include an overlying or overlapping material that is flexible and/or less rigid such as latex, rubber and/or silicone. Desirably, the less rigid surface can be in substantial contact with the skin surface of the breast, while the underlying surface is linked to an underlying support and/or motion stimulator. If desired, the moveable elements may be over molded with a soft rubber or may incorporate fabric-like material on one or more surfaces, or a separate rubber or latex insert or cover may be provided, which may cover only a portion of the element, if desired.

In various additional embodiments, the breast pump system may have components or other attachments that are wearable and/or self-adhesive, including components that may be attached directly to the skin surface of the nipple, areola and/or breast, and/or attached directly to various components of the pumping assembly. In examples, an adhesive (such as sticky patches and/or a tacky adhesive) may be used to affix breast pump components to the breast of a wearer. In examples, the adhesive may be tacky silicon. Another examples is a flexible silicone nipple shield that may be placed over and/or secured to the nipple of an individual, with the nipple shield utilized by the pumping apparatus in various manners. In examples, the nipple shield may include one or more attachment features for securement to a nipple manipulation apparatus, or may be utilized in conjunction with a vacuum or similar apparatus. In some examples, the nipple shield may be obtained in a sterile condition and disposed of after one or more uses.

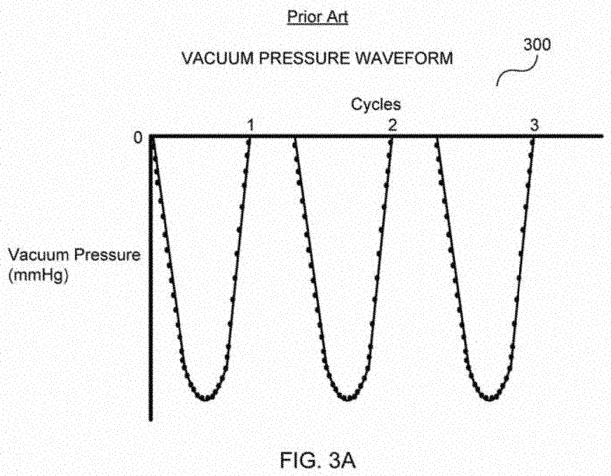

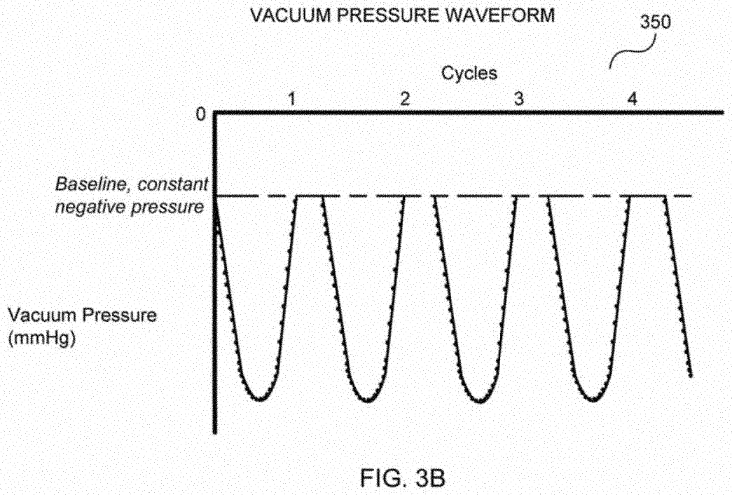

FIGS. 3A and 3B illustrate one comparison of a prior art vacuum pressure waveform 300 that is utilized in prior art breast pumps to an exemplary vacuum pressure waveform 350 which may be employed by the disclosed breast pumping devices and associated components (which may incorporate a baseline suction into the waveform), such as various of the embodiments herein in accordance with various teachings of the invention. In examples, a wide variety of alternative vacuum pressure waveforms may be employed by a vacuum component in the various embodiments, such as vacuum component 122 of FIG. 1, in conjunction with mechanical components 124, such as those discussed in FIGS. 1 and 2.

As best seen in FIG. 3A, the prior art vacuum pressure waveform 300 typically cycles between the surrounding atmospheric pressure (i.e., “0” vacuum on the graph) and an increased vacuum or suction level (i.e., the lower “valleys” in the graph). In particular, commercially available breast pumps generally have vacuum pressure profiles such as shown in FIG. 3A, in which the pressure level periodically oscillates from zero (or equal to the surrounding local atmospheric pressure) to a pre-defined or adjustable negative suction pressure level during each cycle. In addition to drawing milk out of the nipple, many of these prior art devices may utilize the periodic suction force to hold the breast cup in place during suction—a force that is released as the device pressure returns to atmospheric. Absent supplemental fixation or the use of holding devices such as a supportive nursing bra or the user's hands, however, the other breast pumps that utilize such vacuum pressure waveforms 300 may often fall off the breast, dislodge, slide, move and/or lose vacuum seal as the vacuum pressure falls to zero. This tendency can increase where the breast cups include reservoirs for the pumped milk (such as in the breast cups available with the HygieneKit Milk Collection System, commercially available from Ameda corporation of Buffalo Grove, Ill., USA). The tendency for such cups to fall off can further reduce convenience of the system, and the need for supplemental securement can severely limit the amount of movement of nursing women, who may limit their mobility so as to avoid having the breast pump disengage from the breast. Additionally, large oscillations in pressure in such devices may be a source of stretching and friction applied to the sensitive skin at the areola and nipple while pumping, which may cause discomfort.

In contrast, FIG. 3B illustrates an exemplary vacuum pressure waveform 350 with supplemental baseline suction level, which can be utilized in breast pumps and related devices of the invention. In particular, as utilized herein, the vacuum pressure waveform 350 has two primary components: (1) a first component that desirably provides a constant, baseline negative pressure in the breast cups to establish a latch or attachment, and (2) a periodic vacuum waveform from the baseline pressure to a pre-defined or adjustable vacuum pressure level in each cycle, which is then reduced back to the baseline pressure between cycles. The employment of a baseline negative pressure, as described herein, may secure the breast cup or other pump component to the breast, which may improve wearability, comfort, and effectiveness of the breast pump. Specifically, such attachment may enable “hands-free” use of the device, such as hand-free pumping, pumping while nursing, and pumping while sleeping, which are currently difficult with standard suction breast pumps, as most pumps require the user to be in an upright position while pumping as there is no constant suction or other retention features to ensure the breast cup does not leave contact with the breast and/or milk does not spill outside of the cup. Additionally, a second component of the pressure waveform desirably provides a periodic suction waveform from the baseline pressure to a pre-defined or adjustable vacuum pressure level in each cycle. The oscillations of the second component may be used in the existing manner to help extract breast milk from the breast. In various additional embodiments, because the second component of the periodic vacuum pressure waveform 350 can be used in conjunction with an underlying baseline pressure, such as illustrated in FIG. 3B, the degree of oscillations may be smaller, which may lessen friction of the breast pump on the breast compared to breast pumps using a standard vacuum pressure waveform 300. Desirably, the baseline pressure may be sufficient to maintain the connection between the breast and the breast cup, but sufficiently low to allow removal of the cup from the as desired by the user.

In various embodiments, the suction waveform can consist of a constant component and periodic component. If desired, the constant component may be achieved through a smoothly increasing or gradual (i.e., stepwise vacuum increase).

In various embodiments, the constant component may vary in magnitude over time, and/or the periodic component may vary in frequency over time. If desired, the magnitude of suction strength may vary over time during a single period. Magnitude and/or frequency of oscillations may be fixed, user adjustable, or automatically adjusted (i.e., responsive to the user).

If an inconspicuous “hand free” milking and/or stimulating device is desired, the system may include a milk collection vessel placed inconspicuously in a cleft between the user's breasts and/or in a pocket of clothing and/or elsewhere, along with a portable battery-operated pump or battery powered motor.

In some embodiments, the combination of stimulation and suction might facilitate milk production at reduced suction from conventional milk expression machines. For example, various suction oscillation programs may include systems that require a maximum suction ranging only between 0 to 50 mmHg and/or between 50 to 70 mmHg and/or between 70 to 90 mmHg and/or between 90 to 120 mmHg and/or between 120 and 200 mmHg and/or more.

In a first stage of operation, one or more subsystems can be initialized. For example, a user may position the nipple and/or areola stimulator onto the breast, or the stimulator may form a portion of the milk collection apparatus (i.e., secured within the breast cup). Alternatively, the user might secure a brassiere including the stimulators and/or milk collection apparatus. If desired, the initiation of some subsystems may be linked, such as where the stimulator may be installed into a collection flange. The collection flange may optionally be positioned over the nipple and/or areola when the stimulation unit is already being worn.

In various embodiments, an initial baseline pressure may be provided by the vacuum pump or other low pressure source, or alternatively the breast cup and/or vacuum pressure line may include a pressure relief valve or other arrangement that allows the vacuum pump to apply the higher suction pressures to the breast cup (with the pump eventually lowering such vacuum pressure in a standard manner), but which retains a low level of vacuum pressure between suction cycles from the pump.

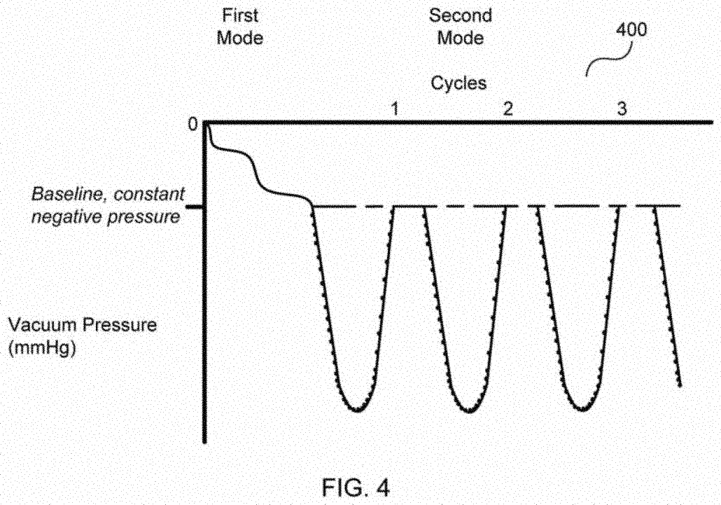

FIG. 4 illustrates another embodiment of a progressive vacuum pressure waveform 400 which may be utilized by various breast pump designs and pumping methods, in accordance with various teachings of the invention. A progressive method of gradually increasing an initial baseline pressure level may be employed in an attempt to comfortably achieve an initial latching of the breast cup or other accessory to the nipple, areola and/or breast. In particular, a progressive waveform may be used to gradually reach a baseline, with a constant negative pressure level in a first mode before beginning a second mode of periodically altering pressure. The gradual, progressive latching may allow a nursing mother to position and orient the breast cup relative to the nipple, areola and/or remainder of the breast in an attempt to engage a comfortable fit. The gradual, progressive latching may also reduce the discomfort that may be felt when a breast pump affixes to a breast with a rapid increase of pressure. Once a proper fit and positioning is achieved, a higher baseline pressure and/or the periodic vacuum cycle previously described may begin. If desired, the system may utilize a sensing device to determine if sufficient baseline pressure has been achieved to initiate the remainder of the suction cycle, or a time delay or manual switch or other feature may be activated by the user once a comfortable fit has been obtained

In various embodiments, the components and systems described herein may be coordinated by a controller or other feature. For example, nipple and/or areolar stimulation may be coordinated with an initial baseline vacuum (i.e., to draw the nipple and areola into the apparatus) before milk collecting is initiated. In such systems, a controller may activate the baseline vacuum and/or initial stimulation of the nipple and/or areola before beginning milk extraction. The controller can optionally be programmed to start milk extraction (for example by activating a higher level of suction of the vacuum pump) and/or change the pattern of stimulation after a period of initial stimulation and/or without a period of initial stimulation. Optionally the user can change the timing, form and/or pressure of the initial and/or subsequent nipple and/or areolar stimulation periods. Alternatively, or additionally, the controller may turn off, turn on and/or adjust setting of the milk extraction pump and/or stimulator in response to sensor output (for example based on measured breast volume and/or extracted milk amount). During a stimulation period, stimulation may be constant and/or intermittent. Milk extraction may be performed before, during and/or after a stimulation period. Milk extraction may be constant and/or intermittent. For example, intermittent extraction may optionally be cyclic depending upon the female's individual “let down” cycles, of which there may be multiples (i.e., four or more) during a single pumping session. If desired, milk extraction may take place during a break between stimulation intervals. Optionally, collecting may be coordinated with tracking. For example, a milk collection bag or other container may serve as a storage and/or collection vessel for the milk. The bag may include a mass sensor, for example a load cell and/or capacitance cell and/or a Wheatstone bridge having a capability of sensing the volume, weight, mass and/or tension of the bag. Measurement results of the mass sensor may be transferred to a controller and/or a tracker, which may include an application operating on a portable computer or mobile phone of the user (which may be linked to various social media sites on the internet, if desired). The mass data may be analyzed by the controller and/or logged to a memory, and an instantaneous and/or time-dependent flow rate may be calculated. When the rate of flow is small an alarm may sound, which may include indicating the end of a milking session (or a transient reduction in milk production) and/or indicating that one or more components of the system may not be working efficiently.

In various embodiments, the collection of milk and the breast stimulation may be coordinated and/or sequential, such as where the stimulation begins and stops, and then milk collection is initiated and then terminated, with the cycles repeating. Compression may be periodic in nature (i.e., similar to the amount that a baby compresses during feeding of 50+ times per minute). The frequency, distance and/or force of compression may be variable over time (and/or over multiple cycles) and might be adjustable by the user or pre-set for comfort and/or efficacy. In various alternative embodiments, the compression cycles it may be automatically responsive to the user—such as where the device may contain a sensor within the milk collection unit or within the path of milk flow to automatically determine the optimal compression frequency and strength. This feature may be especially valuable to some users, as a woman typically experiences multiple milk letdowns during a single breast pumping session. In a manner similar to the breastfeeding baby who adjusts their speed and strength of compression and suction to stimulate letdowns, the system may mimic this approach and use a faster, less forcible suckling to stimulate “letdown without swallows.”

In various additional embodiments, a compression distance and/or force induced by the stimulating elements may be variable over time within a single period, and from point to point along the areola and/or nipple. This design may enable mimicking of the motions such as when the nipple is stimulated by hand, such as when a user commonly squeezes the areola between index and thumb and pulls towards nipple, with greater force as the user moves towards nipple. There may alternatively be multiple modes, or compression profiles, (for example, pinching, squeezing, rubbing) that are user adjustable or automatically pre-set. To enable these differing modes, the bladders/plates/rollers may be moved in varying positions. If desired, plate motion may be combined with a constant or periodic vibration.

FIG. 5 illustrates components of one exemplary embodiment of a breast pump assembly, in accordance with various teachings of the invention. In this embodiment, the breast pump 500 includes an outer cup 505 that fits over the nipple, areola, and portions of the breast. The breast pump 500 also includes an upper plate 510, a lower plate 515, a mechanism 520 to move the lower plate 515 relative to the upper plate 510, a flexible inner liner 525, a liner 530, an adjustable clamp 535 or other attachment feature, and a tube 540 for milk and/or vacuum. The liner 525 may include a heating element, if desired. Various of these features may be incorporated into an inset that fits within the breast cup, or may be connected to, or built into, the outer cup that fits onto the breast (i.e., attached to the cup walls directly).

The shape of the upper plate may resemble the baby's upper palate. In particular, the upper plate may resemble the upper palate of a baby's mouth in size, dimension, shape, and possibly texture and material, that makes contact with the areola. Some examples of this are provided in Figures of this application. The upper plate may be curved. In some examples, the highest point of the upper plate may be closest to the nipple. In some examples, the lowest point of the upper plate may be the first point of contact with the areola. In some examples, the curvature of the upper plate may be used to ensure that when the areola is pressed against the upper plate, the flesh of the breast may expand and/or elongate in the direction of the nipple. In this way, the flesh of the breast may not be pushed backwards, but instead may elongate in the direction of the nipple.

The upper plate may not touch the nipple, but rather may press against only the areola and leave the nipple in free space so that the nipple does not suffer friction during compression motion of one or more breast pump components. The shape of the upper plate may also be optimized to aid in achieving the desired motion profile during compression of the areola.

The various embodiments described herein contemplate a variety of techniques, systems and/or methods for squeezing, stroking, pinching, milking, massaging and/or otherwise manipulating the skin surface and/or underlying tissues of the nipple, areola and/or breast in a desired manner. For example, the mechanism 520 may comprise a mechanism that physically moves and/or displaces the lower plate 515, such as a linear or reciprocating motor or gearing arrangement, a vibratory massage mechanism, a magnetic or hydraulic actuator, a roller arrangement, a pneumatic system such as an inflatable balloon, or some other way of altering the position of the lower plate relative to the upper plate, relative to the outer cup 505 and/or relative to the nipple or areola. Other mechanism arrangements may be employed to move the upper plate 510 in a similar or different manner, which may include linear lotion, rotational motion, peristaltic motion and/or various combinations thereof.

In at least one exemplary embodiment, the one or more plates acting upon the areola and nipple may move in one or more directions via linear motion. The motion of the plates may be powered by an electric motor or an air pump with bellows that enable it to compress the tissue. The plates may be made of varying size, thickness, and/or shape, with a desired direction/distance of motion to enable the desired motion/compression profile on the areola during inflation and deflation. If desired, the motion may be achieved using multiple plates in conjunction or in sequence with one another.

In one alternative embodiment, the one or more plates may comprise one more “rollers” attached to the external circumference of a rotor, such as within the pumping mechanism of a peristaltic pump, to desirably stroke and/or compresses the tissue as the rotor turns. Alternatively, such roller(s) may be attached to a hose, chain, or belt that is arranged like a conveyer belt and is powered by the rotor to achieve linear rather than purely circular motion of the belt. In various arrangements this design may create a peristaltic action to compress, pinch or pull the areola and/or nipple. The diameter or the height of the individual rollers may be adjustable, or fixed and designed to achieve a desired compression profile.

The nipple and/or areolar stimulator may comprise a variety of features, including one or more plates or flexible bladders and/or inflatable bladders or rings to optionally encircle a nipple and/or an areola. Elements may be moved and/or inflated/deflated serially and/or simultaneously. Desirably, the stimulation should mimic stimulation of the nipple and/or areola by the palette and/or tongue of an infant during breast feeding. Nipple and areola stimulation as described herein may express milk on its own and/or may be used in combination with a vacuum pump to extract milk. Optionally, the stimulation devices described herein may be used without a milk extraction unit, for example to help induce and/or accelerate labor.

Various embodiments described herein optionally include one or more stimulation elements for stimulating portions of the breast, including the nipple and/or areola. Stimulation or massage elements may include physical devices (i.e., pads or bodies) as well as pneumatic massage elements (i.e., inflatable bladders or inflatable rings encircling some portions of the end of the breast, including around the areola and/or nipple), or combinations thereof. In various embodiments, some or all of the stimulating elements or rings may be divided into sections that may be controlled separately (i.e., longitudinally and/or radially). For example, some elements or rings may be positioned on opposing sides of the breast tissue therebetween, or connected in a helical form and/or as multiple connected rings. Varying numbers and dispositions of elements or rings may partially surround the breast, for example the elements or rings may surround 50% of the nipple and areola, more than 70% of the circumference of the breast and/or an element or ring may surround more than 90% of the circumference of the breast.

In various embodiments, stimulating elements may be activated by a pump (i.e., via vacuum and/or pressure of air, water or some other gas or fluid), or by movement of the breast cup, or by motorized movement, or movement of the breast or elements thereof may initiate contact with the stimulating elements. If desired, stimulating elements may encircle only a portion of the circumference of the breast. Stimulating elements may optionally be activated simultaneously, sequentially, and/or various combinations thereof, for example to compress the nipple and/or areola.

The various stimulation and milk expression steps may be performed to express milk with a wave like motion. For example, a first stimulation element may be optionally activated in the proximity of the areolar boundary (or outside of the boundary, if desired) compressing breast in a first region near the areolar boundary and/or pushing milk from ducts and/or sinuses towards the nipple pores. Subsequently, a second stimulation element may optionally be activated, compressing breast further towards the nipple pores. Subsequently, a third stimulation element may optionally be activated, compressing the breast further and pushing milk towards the nipple pores. While then third element is activated, the first and/or second elements may be released. Subsequently, a fourth stimulation element may optionally be activated, expressing milk out of the nipple pores. While the fourth element is activated, the remaining elements may be released and the process repeated (which may be before, during and/or after the fourth element is released). Alternatively, a similar process may be accomplished by sequentially inflating stimulating elements, or by the use of a rolling element or a rotating plate.

In various embodiment, the plate elements may include surface features shaped and/or dimensioned to extend inwards towards the breast. If desired, there may be a plurality of such plates evenly spaced round the rim of the cup, which may include an even number of plates. If desired, plates may be of differing sizes and/or shapes, to include a series of 3 or more alternating plates of differing sizes. If desired, plate arrangements may include four plates or eight plates, such as where four longer plates were separated by four shorter plates. Each plate may have a curved or bowl-shaped section, convex towards the inside of the plate, that is positioned to press lightly on the nipple and/or areola when the cup or pump is in position on the breast. Each curved section or bowl shaped section may include surface features such as a number of small bumps, etc.

In the embodiment of FIG. 5, the outer cup 505 may remain affixed to the breast utilizing baseline suction or adhesion, which may include using vacuum from an external source such as by using tube 540 or vacuum from an internal source such as by deformation of the outer cup 505, which may induce a lower pressure area in the vicinity of the nipple (i.e., in a manner similar to a nasal suction device or “snake bite cup”). Additionally, or alternatively, outer cup 505 may remain affixed to the breast utilizing light clamps, such as adjustable clamp 535, on the breast tissue using a spring or other force. This adjustable clamp 535 is described further below. The baseline suction and clamping may serve to mimic a baby's latch onto the breast and may enable greater mobility while using the breast pump system. For example, the breast pump system may be used hand-free during operation. Additionally, the outer cup 505 may house an areola and nipple stimulation assembly. The outer cup 505 may be made of a rigid material having a size and dimension to enclose the stimulation assembly, as well as a nipple area, areola area, and possibly a portion of the breast such as a distal portion of the breast. In particular, the outer cup may provide differing degrees of coverage depending on the size of the outer cup and the size of the breast.

In various embodiments, a stimulation apparatus may be provided to mechanically stimulate the nipple, areola, and/or possibly a portion of the breast, such as a distal portion of the breast. The stimulating apparatus may comprise an upper plate 510, a lower plate 515, and a mechanism 520 to move the lower plate 515 with respect to the upper plate 510. An upper plate 510 of the stimulating apparatus may include shape, size and/or surface features that resemble an upper palate of a baby's mouth in size, dimension, and shape that makes contact with the areola and nipple. In other examples, the upper plate 510 may retain one or more components of the characteristics of the upper palate of a baby's mouth. Further, the upper plate 510 may remain rigid or semi-rigid during operation of the stimulating apparatus and/or the lower plate may move in a longitudinal and/or lateral sliding direction, like a baby's tongue.

The areola stimulator may stimulate some and/or all of the nipple 462 and/or areola 464, or side portions thereof. In various embodiments, stimulation may mimic the movements of the tongue and palate of a suckling infant, for example to further encourage lactation, milk let down and/or release. In various embodiments, the stimulating feature may directly contact the skin of the breast, although in other embodiments elements of the stimulation members may be positioned on the outside of a flexible flange or other milk collection feature, for example by stimulating nipple and/or areola by deforming a flexible body or flange around the breast.

A lower plate 515 may also be provided. In particular, the lower plate 515 may resemble the tongue of a baby's mouth in size, dimension, shape, and possibly texture and material that makes contact with the areola and nipple. The lower plate 515 may also move to stimulate the areola and nipple in a compressing, sliding, rolling, brushing and/or sweeping motion. In particular, a mechanism 520 to move the lower plate with respect to the upper plate 510 may be provided. The motion may oscillate according to a predesignated pattern or may be of variable frequency. The lower plate 515 may have linear or peristaltic movement to simulate the tongue in a baby's mouth and act to gently massage the areola and nipple. In other examples, the lower plate 515 may retain one or more components of the characteristics of the tongue of a baby's mouth. Examples of mechanism 520 that may be used to move lower plate 515 with respect to an upper plate 510 may include an electrically or pneumatically shaft and associated driven motor or and a pneumatic or inflatable membrane that is connected to a suction or pressure supply tube. Desirably, the surface of the plate also serve to massage various portions of the breast to stimulate nerves endings and/or stimulate the release of milk from the breast during pumping. The lower plate may press against the areola and portion of the nipple, but may leave the tip of the nipple in free space so that the nipple does not suffer friction during compression of one or more breast pump components. In examples, the lower plate may not touch the nipple. In examples, the lower plate may sandwich the areola surrounding the nipple, in between the upper and lower plate, and may leave the nipple in free space. There may be a vertical and horizontal component to the motion of the lower plate. In examples, the motion of the lower plate may resemble “squeezing” the nipple (e.g., compressing horizontally and vertically, with the most compression near the nipple, but not touching the nipple). To avoid excessive friction, the lower plate may not rub across the nipple. Instead, in examples, the contact points between the skin of the areola and lower plate may remain constant and the motion may cause the areola to compress or expand. Such examples may be used to mimic the baby's mouth, where the tongue of the baby's mouth moves up and down at various points to create a pressure waveform, but may not “roll” or “rub” the areola.

In use, the woman can place the stimulating apparatus over her breast 12, with her nipple centrally located within the cup and with the neck of the cup resting against her breast. A gentle suction or a mechanical capture feature can then be activated to draw the nipple and areola into the cup, and the plates can then be moved into a desired position gently against the breast tissue. The woman can then start the pumping process by activating the plate motion and/or milk suction, as desired. The plate may desirably produce a reciprocating or other motion of the plate against the nipple and/or areola, which massages the breast tissue in a pleasurable manner, simulating the massaging of the breast by a baby's mouth, thereby initiating the hormonal cascade culminating in “let down” and release by the breast of milk, which can be gathered up by the pump and transferred to a collecting reservoir or bottle.

In various embodiments, a mechanism to draw and/or secure the nipple and/or areola within the stimulation assembly can be provided. The mechanism may desirably allow a user to easily insert, secure and elongate (or “latch”) the areola and nipple within the cup/between the upper and lower surfaces. In many cases it may be desirable to elongate and/or secure the nipple in a desired position and/or orientation in order to maximize the area of contact for nipple/areola stimulation, to reduce the friction on the sensitive areola and nipple during stimulation, or to increase the efficacy of nipple stimulation. It may also aid in securing the device to the breast, and hence improve convenience and/or wearability. It may also help to mimic the sensation of the breast within the baby's mouth.

During breastfeeding, the baby elongates and secures the areola and nipple by establishing a seal between its lips and the areola, and pulling the nipple to junction of the hard and soft palates at the rear of the mouth. In effect, the baby sandwiches the areola between its tongue and gums, and elongates the areola so it is flattened in the vertical dimension and stretched in the horizontal direction. The flesh is tightly sandwiched and it desirably does not create excessive friction on the skin during sucking, which is facilitated by the lubrication provided by the baby's saliva as well as the fluid dynamics of the milk itself.

In various exemplary embodiments, a similar motion of drawing and securing of the breast tissues can be accomplished using mechanical capture devices and/or suction, or a combination of suction and mechanical motion in a particular sequence, to comfortably elongate and/or secure the areola and/or nipple between the nipple stimulation component. For example, a capture device may comprise a ratchet clamp (e.g., clamp 525) that enables expanding and contracting of the opening between the upper and lower surfaces of the areola/nipple stimulation mechanism to comfortably insert and secure (or “sandwich”) the areola and nipple between the surfaces. The distance of compression may be according to user preference or at a fixed distance.

In one alternative embodiment, the outer cup of the breast pump, to which the upper and lower surfaces may be attached, may be made of one or more elastic components that enable it to open like a baby's jaw and snugly secure the areola and nipple between the upper and lower surfaces. In one such embodiment, the opening of the device may be between 21 mm to 27 mm, to ensure the opening captures the areola but does not extend too far beyond the skin of the areola, after which suction pressure required for milk extraction may become uncomfortable on the skin. In alternative embodiment, sizes can vary, depending upon the size and/or shape or the individual's breasts, areola and/or nipples. Elongation of the nipple and/or areola may occur between the upper and lower surface, with the surface compression upon the areola (i.e., by the upper and lower surfaces) causing the nipple and/or areola to expand. Once the breast tissues were inserted into the cup and in between the upper and lower surfaces, and before it is secured/compressed between the surfaces, suction pressure may be applied to pull on the areola and nipple. The upper and lower surfaces may then be pressed together to secure the areola and nipple in the elongated state. This suction pressure may be temporarily applied only to achieve the elongation pressure, or it may be a constant negative pressure that is maintained throughout the breast pumping session. If desired, this suction may be the same constant, negative pressure that is referenced in the previously identified suction profile.

In various embodiments, the user may be required to sandwich the areola by hand prior to inserting it into the opening of the device, as is often done when assisting baby achieve latch during breastfeeding. Once the milking session was completed, the areola and nipple may be released automatically or may be accomplished manually by cracking the seal between the breast and cup using fingers, or by expanding the clamp, opening or shutting a valve and/or turning off suction using manual buttons or removal of power.

In another exemplary embodiment, a liner 530, such as a soft liner, may be optionally connected to the outer cup 505. The liner 530 may enclose the areola and nipple stimulating apparatus. Additionally, an adhesive and/or lubricant may be applied and utilized with the cup, which may be used with or without the use of a liner. The liner 530 may be made of a suitable material to minimize undesirable friction on the breast during operation. The liner may also be made of a suitable material to provide comfort. Additionally, the liner 530 may be made of a single material or may be made of a composition of materials. In other examples, the liner 530 may contain a heating element and/or massage element and/or may become warm so as to increase comfort on the breast. The warming and/or warmable material may be used to help stimulate lactation. For example, applying warmth may reduce stimulation time.

Other examples of a liner 530 may comprise a self-adhesive material, such as a silicon liner, that may form a seal on the outer edge of the cup and gently affixes to skin. In these examples, liner 530 may include an outer sealing liner. Further, annular sealing channels may be provided on one or more perimeter elements of the breast cup.

In the disclosed embodiment, a vacuum port may be provided at the end of the outer cup 505. The vacuum port may include a valve, which may be a standard gate or pivot valve or may include reverse flow inhibiting and/or pressure relief features. If desired, the vacuum port may connect to a plastic tube that may be used to apply vacuum pressure to the nipple in order to latch the breast cup to the breast, extract milk and/or transfer extracted milk to a temporary or longer-term storage reservoir. In various embodiments, the vacuum port may also connect to a plastic tube to channel extracted milk from the nipple for collection. The tube channeling milk may be disposable. Alternatively, there may be a single tube coming from the breast cup that has a tube within a tube, whereby vacuum pressure flows only within a first tube and milk flows only within the second tube. As such, the breast pump may be designed to ensure that milk is directed towards the milk tubing and avoids entering the vacuum tubing.

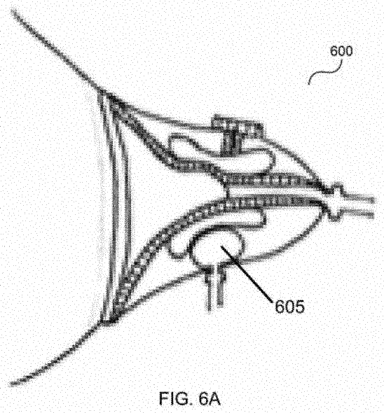

FIG. 6A illustrates exemplary components of a breast pump 600 having an inflatable bladder 605, in accordance with another alternative embodiment of the invention. In particular, as seen in FIG. 6A, the inflatable bladder 605 may be inflated and deflated so as to move the lower plate with respect to the upper plate and/or the nipple. Alternatively, the inflatable bladder may be postponed on the skin side of the lower plate (not shown) with the expandable surface of the bladder contacting the nipple surface directly.

For example, one or more inflatable bladders that inflate and deflate may be utilized to apply pressure to the areola and/or nipple. Each bladder may be connected to an air pipe and/or vacuum pump individually, or may be connected to a distribution system. The various bladders may be made of varying sizes, thicknesses and/or shapes to enable the desired motion/compression profile on the nipple during inflation and deflation, which may include multiple bladders of differing sizes, thicknesses and/or shapes. To achieve a particular desirable motion, such as squeezing, the surface of the bladders may have varying thickness or release holes, or the motion may be achieved using multiple bladders in conjunction or in sequence with one another.

In one alternative embodiment, as shown in FIG. 6B, a motor 610 may be engaged so as to move the lower plate towards and/or away from the upper plate of a breast pump 650. Alternatively, the lower plate may move laterally or other directions, as desired. In various embodiments, the resulting motion of the upper and/or lower surfaces (i.e., induced by the bladders, plates, or rollers) on the areola and nipple may be periodic or cyclic in nature.

In order to mimic the nipple stimulation of a baby, a much larger portion of the areola may be involved in the stimulating apparatus of the invention, which may include the compression and/or encompassing of a large amount of the nipple and/or areolar tissue within the breast pump and/or accessories than is currently employed by traditional pump funnels. In other embodiments, it is contemplated that the breast pump mechanism may include the compression and/or encompassing of a smaller amount of the nipple and/or areolar tissue within the breast pump and/or accessories than is currently employed by traditional pump funnels. Desirably, various embodiments described herein may result in a breast pump mechanism of significantly smaller size, weight and/or complexity than current pump devices, with desirably a commensurate lowered power requirement for operation. In various embodiments, a truly portable and easy to use breast pumping and milk storage mechanism is described.

The present embodiments desirably replicates a more natural milk collecting environment, where the woman can produce and store milk while reclining, sleeping, or walking, and need not necessarily have to remain stationary or upright during pumping. Desirably, a pumping device should be able to extract, collect and channel milk into a collection without spilling with the user at varying positions, which may be achieved through judicious use of the disclosed suction waveforms and/or incorporation of the proper mechanical components. For example, this may be achieved or aided through the constant baseline suction that extends continuously from where milk is released to the collection unit (including through interconnecting pipes and check valves).

In various alternative embodiments, the pumping and collection device may mimic the swallowing mechanism in the mouth and throat of a baby, and capture the milk at the source of extraction (i.e., the nipple pores) into a spill-proof hose, and use a peristaltic motor to capture the fluid and then channel it through one-way fluid valves that extend to the milk collection unit. In such a case, the constant or periodic suction to extract milk along with mechanical motion by the peristaltic pump might be sufficient to push it through the one-way valves. For example, in the case that the upper and lower surfaces of the areola/nipple stimulation device are powered by a peristaltic action or similar drive system, the same mechanism may be used to also capture the liquid and/or channel it into the milk collection unit.

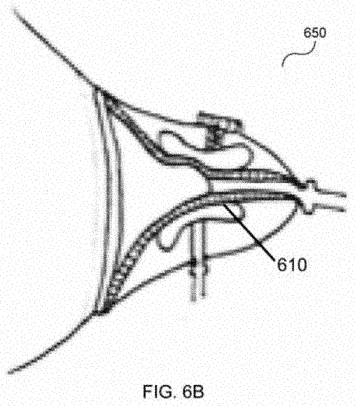

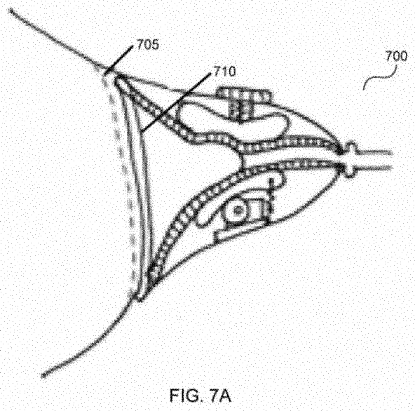

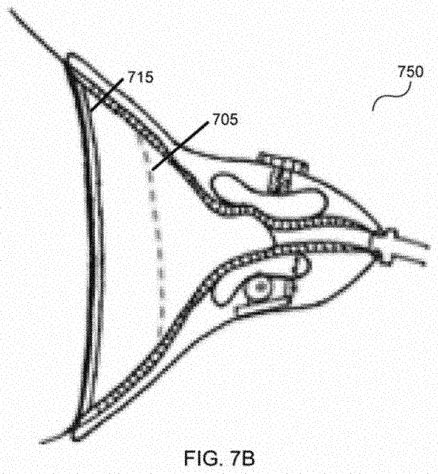

FIG. 7A illustrates components of another alternative embodiment of a breast pump 700, with features and components arranged to attach primarily around an areola and nipple of a breast, in accordance with various teachings of the invention. In this embodiment, breast pump 700 desirably attaches and/or secures to portions of the breast such that the outer edge 710 of the breast cup of the breast pump is approximate to and/or within the areola boundary 705. As illustrated in FIG. 7A, breast pump 700 affixes and compresses a large portion of the areola, forms a seal, pulls the areola and/or nipple into position such that a distal portion of the areola is held between an upper plate and a lower plate, and compresses the areola to varying degrees. The compression may be symmetric or may not be symmetric. In some examples, the areola may be held between the upper plate and the lower plate such that the areola is compressed to approximately 70% of its original height and expands horizontally approximately 20%. Exemplary illustrations of nipple and/or areolar compression are seen in FIGS. 11A and 11B. Additionally, the compression and periodic suction of the areola utilizing breast pump 700 may be concurrent and/or “in phase.” Alternatively, the compression and periodic suction may not be in phase in equal measure and in some examples the compression and periodic suction may be controlled separately.

In another alternative embodiment, as best seen in FIG. 7B, components of a breast pump 750 can be arranged to attach primarily at a distal portion of a breast, in accordance with various teachings of the invention. In particular, FIG. 7B provides an example of a breast pump 705 that attaches to the breast such that the outer edge 715 of the breast cup of the breast pump is beyond and/or outside of the areolar boundary 705. In this way, FIG. 7B illustrates components of an additional embodiment of a breast pump that attaches primarily to breast tissues beyond an areola.

FIG. 8 illustrates components of another alternative embodiment of a breast pump cup 800 having multiple vacuum ports 810, 815, constructed in accordance with various teachings of the invention. In this embodiment, an areolar stimulation component 805 is provided, which can optionally apply movement relative to the upper plate and/or outer cup with variable motion, variable frequency and/or variable strength. The stimulation being provided may be compressive, rolling, rocking and/or sliding, which may include pre-defined stimulation and/or stimulation that is adjustable by the user. In various embodiments the stimulation may be operated independently without associated suction, or the stimulation may be operated in conjunction with suction, or stimulation and suction can be operated sequentially. The stimulation may also be automatically adjusted in real-time by the device, including according to feedback and/or measurements that may be collected from a controller within the apparatus.

FIG. 8 also depicts a suction line attached through a front vacuum port 810, although in various other embodiments the positioning of vacuum port 810 need not necessarily be at the tip of the cup 800. Suction provided through vacuum port 810 may provide a baseline constant negative pressure and/or a suction pressure to initially draw the nipple and/or areola into the cup 800. The baseline constant negative pressure may be of variable strength, may be pre-defined, may be adjustable by the user, or may be achieved with progressive and/or gradual waveform (including those waveforms previously described herein). The front vacuum port 810 may also provide periodic suction, if desired. The periodic suction may have variable frequency and/or variable strength. Additionally, the periodic suction may be pre-defined, may be adjustable by the user, may be in conjunction with nipple massage, may be without nipple massage, or may be in conjunction with a baseline negative pressure. The suction may also be automatically adjusted in real-time by the device, according to feedback collected from a controller within the apparatus.

FIG. 8 also depicts an outer seal arrangement that can include a supplemental attachment or vacuum component, provided by a suction or vacuum passing through vacuum port 815. The outer seal may include an annular sealing chamber utilizing vacuum pressure of variable strength, which may be of the same or a reduced pressure as compare to the suction through the vacuum port 815. The outer seal may also be composed of a suitable material that forms a seal with the skin, such as silicon or similar materials. If desired, the cup 800 may include a clamp component 820, which can utilize spring force, may be at a fixed distance, may be automatically adjusting, or may be adjustable by a user to capture and/or retain the nipple and/or areola in a desired manner.

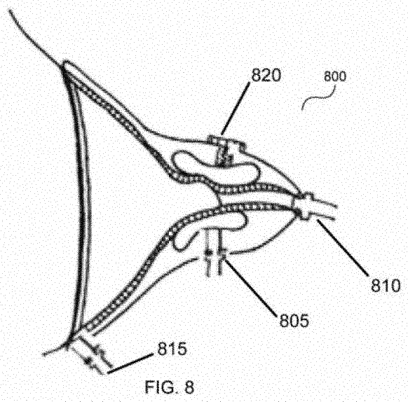

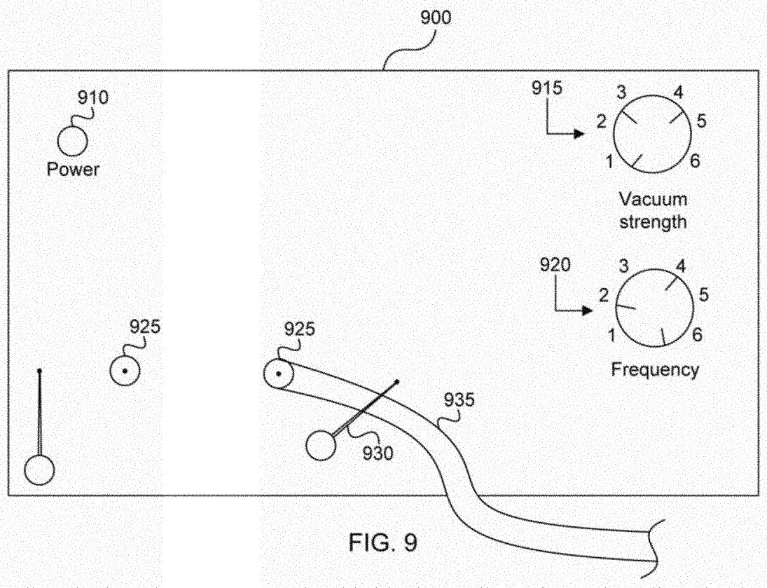

FIG. 9 illustrates one exemplary embodiment of a vacuum source control panel 900, in accordance with various teachings of the invention. In particular, the control panel 900 includes a power button 910, a dial 915 that may be used to adjust vacuum strength, a dial 920 that may be used to adjust vacuum wavelength frequency, vacuum sources 925, vacuum source covers 930, and tubing 935 that may be used to provide vacuum pressure to a breast pump. Vacuum source covers 930 may be used to cover vacuum sources 925 so as to prevent outside contaminants from entering the vacuum sources 925. Additionally, the vacuum source covers 930 may be used to keep one vacuum source 925 closed when there is only one vacuum source 925 in use, such as seen in FIG. 9. As such, prior to engaging the vacuum source 900, a vacuum cover 930 may be placed over the vacuum source 925 that is not attached to a vacuum tube 935.

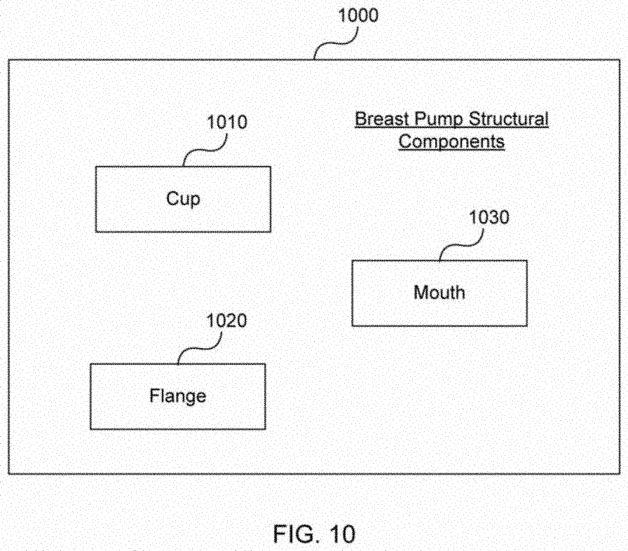

FIG. 10 provides a block diagram of one embodiment of various breast pump structural components 1000, in accordance with teachings of the invention. In particular, breast pump structural components 1000 can include components of a breast pump that come into contact with the breast. A cup component 1010, such as outer cup 505, may be used to contain a nipple area, an areola area, and optionally a portion of the breast. Additionally, the cup component may have a flange 1020 that extends from the cup to a portion of the breast and optionally a portion of the areola. The design and extension of flange 1020 may be determined based on factors such as comfort, wearability, and ease of use. Additionally, cup component 1010 may be used to house a mouth component 1030 that is formed from portions of a stimulation assembly, such as the stimulation assembly discussed in FIG. 5.

FIGS. 11A and 11B provide an illustration of compression within a stimulation mechanism, in accordance with embodiments of the invention. In particular, FIG. 11A provides a stimulation mechanism at a first position, with an upper plate engaging a top portion of a nipple area and areola area and a lower plate engaging a bottom portion of the nipple area and areola area. Additionally, FIG. 11B provides a stimulation mechanism at a second position, with the upper plate further compressing the top portion of the nipple area and areola area and the lower plate further compressing a bottom portion of the nipple area and areola area.

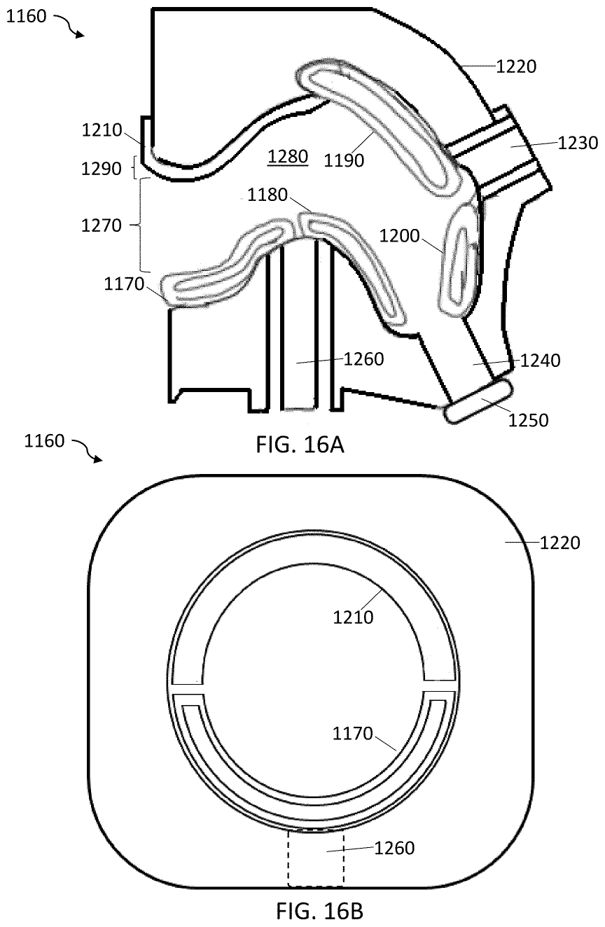

FIG. 16A-16B depicts a cross-sectional side view and a front (or anterior view) of an alternate embodiment of a breast pump 1160. The breast pump 1160 comprises a cup or a body 1220, a stimulating mechanism, and a vacuum mechanism. The breast pump 1160 may further comprise a computer system, the computer system includes at least one of the following: display screen, microprocesser, a memory (e.g., RAM or ROM), a network interface and/or any combination thereof. The body or cup 1220 may further comprise a plurality of ports 1230, 1240, 1260 and a cavity 1280. The plurality of ports 1230, 1240, 1260 may be spaced apart and oriented in different directions. The plurality of ports 1230, 1240, 1260 may comprise a vacuum port or a milk port or milk drain. The plurality of ports 1230, 1240, 1260 and/or each of the plurality of ports 1230, 1240, 1260 may further comprise a cap or a lid 1250. In one embodiment, the body or cup 1220 may comprise a first port 1240 and a second port 1260. The first 1240 and/or the second 1260 port may comprise a pressurized port, a vacuum port or a milk port. The body or cup 1220 may further comprise a third port 1230. The third port 1230 may comprise a vacuum port. The cavity 1280 may be in contact with breast milk. A vacuum port may deliver pressurized air (e.g., to inflate balloon elements), deflate or produce an air pressure that is less than atmospheric pressure.

The cavity 1280 may match or substantially match the cavity opening of an infant during breast feeding. The cavity 1280 may have a first portion and a second portion. The first portion may be positioned anteriorly, the second portion may be positioned posteriorly. The first portion may extend anterior to posterior in an oblique orientation. The second portion extends downwardly and in the oblique direction. The second portion may be in direct communication with at least one port 1230, 1240, 1260 and/or the first port 1240.

The stimulating mechanism may comprise a first or upper element 1210 and a second or lower element 1170. The stimulating mechanism may simulate mouthing sequence of an infant (e.g., squeezing out the nipple and areolar contents by compression between the first element 1210 and lower element 1170) and/or the sucking sequence of an infant (e.g., emptying of the nipple-areolar contents by development of cyclic sub atmospheric pressures while between the first element 1210 and the second element 1170). The upper or first element 1210 that may simulate a baby's upper palate 1050, 1060; the lower or second element 135 may simulate a baby's tongue 1120. The first element 1210 may comprise a first material, the first material matching or substantially matching the elasticity and/or durometer of a baby's upper palate 1050, 1060. The second element 1170 may comprise a second material, the second material matching or substantially matching the elasticity and/or durometer of a baby's tongue 1120.

The second element 1170 is desirably moveable relative to the first element 1270 along one or more directions and one or more frequencies. The movement of the second element 1170 may comprise an opening and closing movement, a peristaltic or undulated movement, a linear movement, and/or any combination thereof. The one or more direction may comprise vertical direction, a horizontal direction, anterior to posterior direction, an oblique direction, and/or any combination thereof. The one or more frequencies may comprise at least 1 Hz or greater; at least 1 Hz to 2 Hz; 1 Hz to 3 Hz. The one or more frequencies may be adjustable by the user of at least 0.25 Hz increments or greater.

In one embodiment, the stimulating mechanism may comprise a first element 1210 and a second element 1170. The second element 1170 is moveable relative to the first element 1270 from a first position to a second position in a first movement type, a first direction and a first frequency. The first movement type may comprise opening and closing of the first element 1210 and the second element 1170. The first element 1210 disposed within the cup 1220 near, adjacent or proximate to the anterior end of the cup 1220. The first element 1210 having a portion of a first surface being in contact with a portion of the areolar 1008 and/or nipple 1015. The second element 1170 disposed within the cup 1220 opposite of the first element 1210, and having a second surface being in contact with a portion of the areolar 1008 and the nipple 1015. The second element 1170 is spaced apart with a first spacing 1270 from the first element 1210 at the first position, which the first position or the first spacing mimics the opening, the first spacing may comprise 25 mm in width up to 50 mm in width.

The second element 1170 is moveable to a second position, which simulates the closing, the second element 1170 is spaced apart with a second spacing (not shown) from the first element 1210, the second spacing being less than the first spacing. The second spacing being less than the first spacing comprises the second spacing being at least 4 to 5 mm difference or 4 to 5 mm less than the first spacing. Accordingly, the opening and closing of the first movement comprises opening and closing in the vertical direction. The movement of the second element 1170 from a first position with a first spacing to a second position with a second spacing occurs in the vertical direction. The second position or the second spacing further mimics the closing, the second spacing or second position may comprise 20 mm to 45 mm in width. The first frequency may comprise at least 1 Hz or greater; at least 1 Hz to 2 Hz; 1 Hz to 3 Hz. The one or more frequencies may be adjustable by the user of at least 0.25 Hz increments or greater. The first movement may simulate the mouthing sequence of an infant (e.g., squeezing out the nipple and areolar contents by compression between the first element 1210 and lower element 1170).

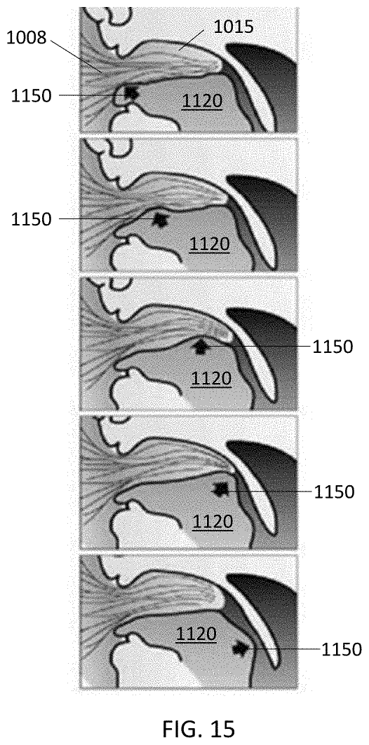

In another embodiment, the stimulating mechanism may further comprise a first element 1210 and a second element 1170 with a second movement. The second element 1170 is further moveable relative to the first element 1270 from a first position to a second position in a second movement type, a second direction and a second frequency. The second movement type comprises a peristaltic movement or motion and/or an undulated movement or motion. The peristaltic movement comprises waves of compression or undulations that moves along the length of the nipple 1015 as shown in FIG. 15. The peristaltic movement may further comprise waves of compression that moves along the length of the nipple 1015 while the compressing the areolar 1008 and nipple 1015, which the second element 1170 moves from a first position to a second position with a second spacing. The second direction occurs in a horizontal direction, from anterior to posterior position within the cup 1220. The resulting action of the peristaltic action of the second movement from the first end or anterior end of the second element 1170 to the second end or posterior end of the second element 1170 simulates pushing the milk back towards the baby's mouth. The posterior end or second end of the second element 1170 is near, adjacent or proximate to the at least one port 1230, 1240, 1260. The at least one port 1230, 1240, 1260 may comprise a milk port or milk drain. The second direction of the second element 1170 from a first position to a second position occurs in the horizontal direction and/or anterior towards posterior end. The second frequency may comprise at least 1 Hz or greater; at least 1 Hz to 2 Hz; 1 Hz to 3 Hz. The one or more frequencies may be adjustable by the user of at least 0.25 Hz increments or greater. Alternatively, the peristaltic motion may comprise a delay, the delay includes 0.10 seconds to 0.20 seconds; 0.10 seconds to 0.15 seconds and/or 0.13 seconds to 0.15 seconds. The total cycle may terminate at between 0.62 seconds to 0.65 seconds or at least 0.64 seconds or greater.

In another embodiment, the stimulating mechanism may further comprise a first element 1210 and a second element 1170 with a third movement. The second element 1170 is further moveable relative to the first element 1270 from a first position to a second position in a third movement type, a third direction and a third frequency. The third movement type may comprise a linear oscillation movement, and/or a back-and-forth movement. The third direction may comprise a linear oscillation movement in a horizontal direction or anterior to posterior direction. The third frequency may comprise at least 1 Hz or greater; at least 1 Hz to 2 Hz; 1 Hz to 3 Hz. The one or more frequencies may be adjustable by the user of at least 0.25 Hz increments or greater.

In another embodiment, the stimulating mechanism may further comprise a third element 1180. The third element 1180 may be positioned adjacent or proximate to the second element 1170. The third element 1180 may represent a portion of a tongue 1120. The third element 1180 may be moveable from a first position to second position relative to the first element 1210. The third element 1280 may be further moveable from a first position to a second position relative to the second element 1210. Alternatively, the third element 1280 may be a continuation of the movement from the second element 1170. Alternatively, the third element 1180 may represent the posterior portion of the tongue.

The stimulating mechanism may further comprise a fourth element 1190. The fourth element 1190 may be positioned adjacent or proximate to the first element 1210 and/or the third element 1180. The fourth element 1190 may be movable from a first position to a second position relative to the second element 1170. The fourth element 1190 may be movable from a first position to a second position relative to the third element 1180. The fourth element 1190 may be movable from a first position to a second position relative to the first element 1210, the second element 1170, the third element 1180, and/or any combination thereof.

The stimulating mechanism may further comprise a fifth element 1200. The fifth element 1200 may be positioned adjacent or proximate to the third element 1180 and the fourth element 1190. The fifth element 1200 may be movable from a first position to a second position relative to the second element 1170. The fifth element 1200 may be movable from a first position to a second position relative to the third element 1180. The fifth element 1200 may be movable from a first position to a second position relative to the fourth element 1190. The fifth element 1200 may be movable from a first position to a second position relative to the first element 1210, the second element 1170, the third element 1180, the fourth element 1190, and/or any combination thereof.

The first element 1210 may comprise a material. The material may match or substantially match the flexibility and elasticity of an infant's soft palate. The material may match or substantially match the flexibility and elasticity of an infant's hard palate. The first element 1210 may comprise a first portion and a second portion. The second portion may comprise a material that matches or substantially matches the flexibility and elasticity of an infant's soft palate. The first portion may comprise a material that matches or substantially matches the flexibility and elasticity of an infant's hard palate. The first portion material may be different than the second portion material. Accordingly, the material may be a flexible plastic or rubber material. The flexible plastic may comprise a thermoset elastomer or a thermoplastic elastomer.

The first element 1210 may further comprise a first portion or a first end and a second portion or a second end. The first portion or first end having a first width and a second end or second portion have a second width. The first width being larger than the second width. The first width comprises at least 1.9 mm. The first portion or the first end is positioned at or within or adjacent to the anterior position of the cup 1220. The first portion or the first end is position within or adjacent to areolar 1008 and/or the breast 999.

The second element 1170 may further comprise a first portion or a first end and a second portion or a second end. The first portion or first end having a first width and a second end or second portion have a second width. The first width being smaller than the second width. The second width comprises at least 3 mm. Alternatively, the second element 1170 may be positioned at an angle or an oblique orientation. The angle may comprise a range of 5 degrees to 30 degrees. In another embodiment, the second end may be positioned higher or taller than the first end of the second element 1170. The second end of the second element 1170 may be at least 3 mm taller than the first end of the second element.

The second element 1170, the third element 1180, the fourth element 1190, and/or any combination thereof, may comprise an inflatable balloon. The inflatable balloon may be movable from a first position to a second position. The first position being at rested position or deflated, and the second position being an inflated position to inflated distance. The inflatable balloon comprises at least one chamber. The inflatable balloon comprising a plurality of chambers. Each of the plurality of chambers may be inflated and deflated independently. The inflation pressure may comprise at least 5 psi or greater and/or 10 psi or greater. Alternatively, the second element 1170, the third element 1180, the fourth element 1190, and/or any combination thereof, may comprise a one or more pads and/or one or more plates. The inflatable balloons may comprise a material known in the art to allow easy inflation and deflation.

### Methods of Use

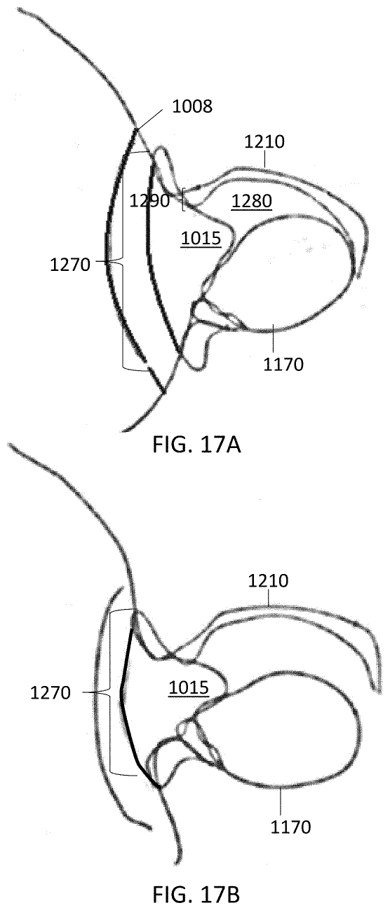

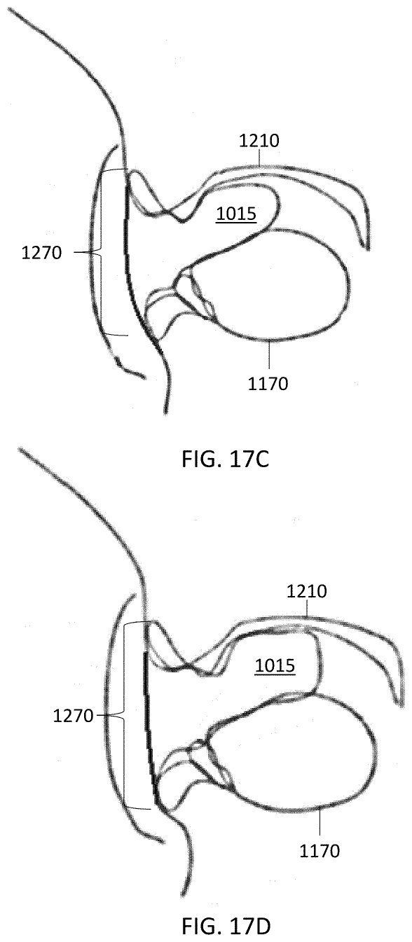

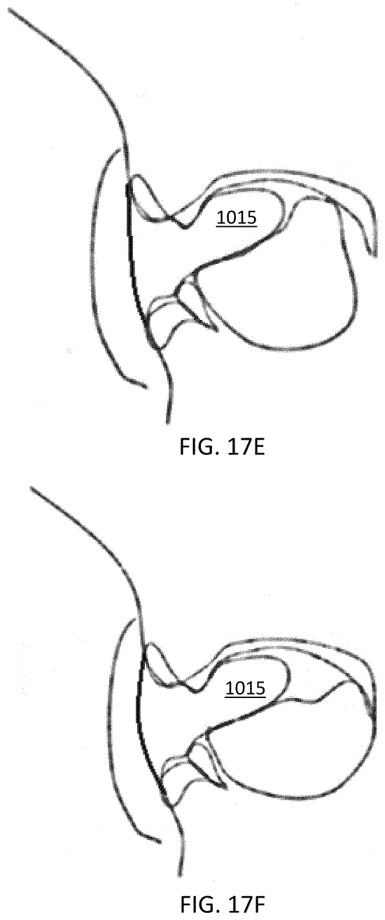

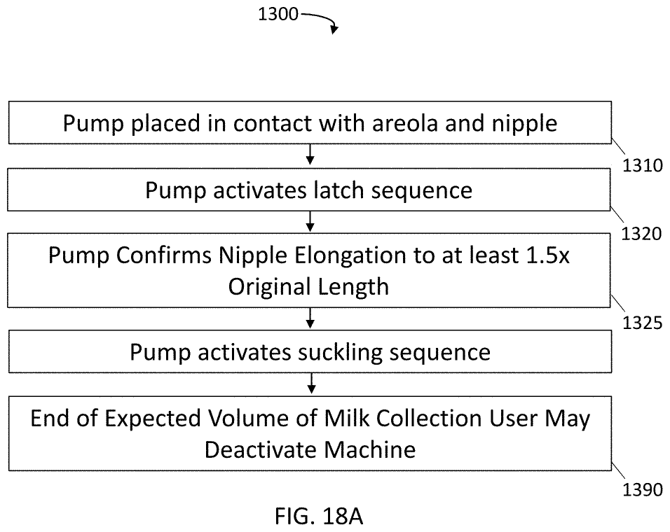

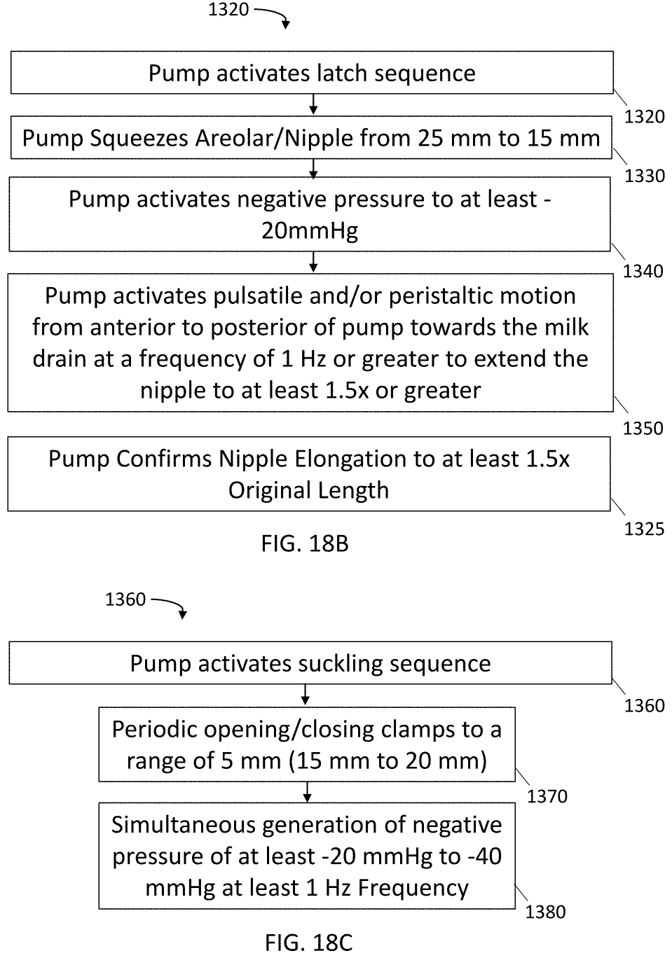

FIGS. 17A-17F depicts a side view of one embodiment of the latch and swallow/suck sequence with simplified view of the plates/pads of the breast pump. In one embodiment, the pump may comprise the steps of activate a latch sequence 1320, and a suckling sequence 1360 as shown in FIGS. 18A-18C. The pump may further comprise activation of a swallow sequence. Each of these sequences perform in way to simulate natural breast feeding steps from an infant. The pump may elongate, flatten and stimulate the areola 1008 and the nipple 1015 in a very specific shape and method to maximize milk extraction. The first step 1310 places the pump cavity opening 1280 in contact with the areolar 1008 and the nipple 1015. The cavity opening 1280 is sized and configured larger than an average diameter of a nipple 1015. The average diameter of a nipple 1015 may comprise at least 12 mm or greater. The nipple 1015 may extend freely into the cavity 1280.

The latch sequence 1320 may comprise one or more of the following steps squeezing or compression 1330, initial vacuum pressure 1340, and/or peristaltic motion 1350. The latching sequence 1320 simulates the infant latching onto the breast and nipple so that the nipple 1015, areola 1008, and underlying mammary tissue and lactiferous ducts 1005 are drawn into the infant's mouth with the nipple tip extended as far as the hard-soft palate junction and/or the center of the cavity 1280 of the cup 1220. Once the latch sequence 1320 is activated, the pump will activate compression cycle 1330, which squeezes a portion of the areolar 1008 and the nipple 1015. The simplified pump as shown in FIGS. 17A-17F comprises a cavity 1280, a first element 1210 and a second element 1170 being spaced apart at a first spacing. The first spacing may comprise at least 25 mm in width. The cavity opening at the first end or the anterior end may be sized at least 25 mm. The second element 1170 is movable relative to the first element 1210 in a vertical direction to squeeze or compress the areolar 1008 and nipple 1015. The second element 1170 moves to a second position with a second spacing, the second spacing is less than the first spacing. The second spacing may comprise at least 20 mm or less. Alternatively, the second element 1170 is movable from a first position at a 25 mm spacing, to a second position at a 20 mm spacing. In another embodiment, the second element 1170 is movable from a first position at a 25 mm spacing, to a second position at a 15 mm spacing.

The latch sequence will further activate an initial negative pressure or a vacuum 1340 of at least 20 mmHg to seal the areolar 1008 to the pump cavity 1280. Once the initial negative pressure or vacuum is sustained, the latch sequence will further activate the pulsatile or peristaltic motion 1350 from the anterior end to the posterior end of the second element 1170 at a set frequency. The frequency may be set at 1 Hz or greater. During this step, the second element 1170 may comprise one or more chambers that are inflated or deflated to elongate the nipple 1015 at least 1.5× and/or 2× or greater than its original size (e.g. 18 mm to 25 mm). Once the nipple 1015 reaches the optimal length, the suckling sequence may be activated.

The suckling sequence 1350 may comprise one or more of the following steps periodic opening and closing oscillations 1370, and negative pressure oscillations 1380. The suckling sequence 1350 mimics or simulates an infant moving its mandible up and down, compressing the areola 1008 and the underlying lactiferous ducts 1008 with its gums in a suckling process that extracts the milk into its mouth or within the cavity 1280 of the cup 1220. The opening and closing oscillations 1370 are disclosed herein. The second element 1170 is movable relative to the first element 1210 in a vertical direction to squeeze or compress the areolar 1008 and nipple 1015 at a set frequency. The second element 1170 moves to a second position with a second spacing, the second spacing is less than the first spacing. Alternatively, the second element 1170 is movable from a first position at least at a 25 mm spacing, to a second position to at least a 20 mm spacing. In another embodiment the second element 1170 is movable from a first position with a first spacing to a second position to a second spacing, the second spacing being at least a 4 to 5 mm less than the first spacing. The set frequency by set at 1 Hz or greater.

Simultaneously, the suckling sequence activates the negative pressure oscillations 1380. The negative pressure oscillations 1380 engages in the change of negative pressure at a set frequency. The negative pressure is movable from a first negative pressure to a second negative pressure, the second negative pressure different than the first negative pressure. The change of negative pressure may occur in a range of 20 mmHg to 50 mmHg; the change of negative pressure may occur in a range of 20 mmHg to 40 mm Hg.

In one embodiment, the steps of using the breast pump assembly 1300 comprises the steps of: contacting areolar and nipple 1310; activating the latching sequence 1320, confirming nipple has elongated to at least 1.5 times its original length 1325; activating the suckling sequence 1360; and deactivating pump after expected milk extraction volume 1390.

The latching sequence 1320 further comprises the step of squeezing or compressing the areolar and nipple from a first spacing to a second spacing 1330, the second spacing being smaller than the first spacing. The latching sequence 1320 further comprising the steps of activating the initial negative pressure to seal the areolar 1340, the initial negative pressure comprises at least −20 mmHg. The latching sequence 1320 further comprises activating the pulsatile or peristaltic motion 1340, the peristaltic motion moves from a first position at the anterior end to a second position at the posterior end at a set frequency.

The suckling sequence 1360 further comprises the steps of activating the opening and closing oscillations 1370 and activating the negative pressure oscillations 1380. The opening and closing oscillations 1370 allow the second element 1170 to move relative to the first element 1210 from a first position to a second position at a set frequency. The negative pressure oscillations allow the pump to change from a first negative pressure to a second negative pressure, the second negative pressure to be different than the first negative pressure. A first negative pressure may comprise at least −20 mmHg and a second negative pressure may comprise at least −40 mmHg. Simultaneous action of latching and suckling sequence may be activated at the same time after confirmation of nipple elongation.

### Breast/Nipple Stimulation or Sex Toy

In various embodiments, the breast pump assembly and/or associated devices may be utilized for purposes other than milk production, such as a stimulation device or sex toy. A sex toy is an object or device that is primarily used to facilitate human sexual pleasure. Many of these devices are designed to resemble human genitals. Sex toys may be vibrating or nonvibrating. Vibrators and other motion devices are used to excite nerve endings in the target regions of the user, and they can be used for masturbation or as part of sexual activities with a partner. Vibrators and other motion producing devices may be used upon the nipples either discretely or in some instances in combination through multiple vibratory elements within the same vibrator or through using multiple vibrators.

In various embodiments, the simulation devices may be utilized alone or in combination with various other components described herein, including the milk expressing, collecting and/or controlling elements.

### Massage or Physical Therapy Device

In some embodiments, the breast pump and/or associated devices described herein may serve additional and/or alternative functions other than enhancing and/or promoting milk extraction. For example, a breast pump may be used to treat engorgement, plugged ducts, and/or mastitis. Other uses may include using the breast pump to push fluid in the lymph system out of the breast. Stimulating elements may be expanded and/or inflated simultaneously, for example to compress breast tissues, which may push fluid in lymph nodes out of the compressed breast tissues. In various embodiments, a local swelling and/or local pain in a breast may be treated by focusing the breast pump on the swollen and/or painful location. For example, stimulation of breast tissues may include expansion/inflation and/or repeated motion (or inflation and deflation) and/or vibration of a stimulating element near the affected area. If desired, the stimulating elements and/or other portions of the breast cup may include a protrusion, finger, leaf and/or ring that partially surrounds the breast. Alternatively, subsonic and/or ultrasonic vibrations may be applied to the area, including the use of focused message elements (for example an inflatable element and/or a vibrator) within the breast cup that can be positioned at an area needing focused massaging.

In some embodiments, a breast may be stimulated and/or pumped to potentially induce labor, to treat the breast for example after surgery, to strengthen breast muscles, to address flat or inverted nipple issues, and/or to shape the breast for aesthetic reasons (i.e., used in a manner similar to a penile pump), to desirably enhance breast and/or nipple size through stimulation). In various alternative embodiments, the breast pump assembly and/or associated devices may be utilized for passive nerve stimulation, massage therapy or as a physical therapy device, such as to restore scarified tissues.

### Mobile Apps

In various embodiments, computer programs or “mobile apps” may be utilized in conjunction with the various devices described herein, such as to monitor pump performance and/or milk collection, share user-created pump control profiles between mothers or other individuals and/or to control or modify the operation of various devices.

While preferred embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the descriptions provided herein.

Many of the aspects and advantages of the invention may be more clearly understood and appreciated by reference to the accompanying drawings. The accompanying drawings are incorporated herein and form a part of the specification, illustrating embodiments of the invention and together with the description, disclose the principles of the invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein.

1. A breast pump, comprising:
an outer cup for accommodating at least a distal portion of a breast,
a first element positioned within the cup, the first element connected to an inner surface of the outer cup and including a generally first rigid base and a first surface for engaging with a first skin surface of the breast,
a second element positioned within the cup, the second element moveable with respect to the first element and including a second generally rigid base and a second surface for engaging with a second skin surface of the breast, and
the first surface of the first element including a curved surface shaped and configured to emulate a baby's upper palate, and the second surface of the second element including a surface shaped and configured to emulate a baby's tongue, the first and second surfaces being spaced apart and facing towards each other,
wherein, when the breast pump is placed over the distal portion of the breast, at least a portion of the breast tissue is positioned between the first and second elements, and movement of the second element relative to the first element causes the first and second surfaces to engage the first and second skin surfaces.

2. The breast pump of claim 1, wherein the movement of the second element cycles between a first position and a second position, a first spacing between the first and second surfaces when the second element is in the first position being less than a second spacing between the first and second surfaces when the second element is in the second position, the first and second surfaces compressing at least a portion of the breast tissue therebetween when the second element is in the first position.

3. The breast pump of claim 1, further comprising a moveable linkage connecting the second element to the outer cup, the moveable linkage including a motor and a controller element, the controller element capable of controlling a movement speed of the second element.

4. The breast pump of claim 1, further comprising a moveable linkage connecting the second element to the outer cup, the moveable linkage including a motor and a controller element, the controller element capable of controlling a movement path of the second element.

5. The breast pump of claim 1, wherein the first element comprises a flexible liner, the flexible liner including a first end and a second end, the first end having a first width and the second end having a second width.

6. The breast pump of claim 5, wherein the flexible liner comprises a heating element.

7. The breast pump of claim 5, wherein the flexible liner comprises a first width being greater than the second width.

8. The breast pump of claim 1, wherein the first surface comprises a generally soft, flexible material.

9. The breast pump of claim 1, The breast pump of claim 1, wherein the movement of the second element comprises peristaltic action from a first end of the second element to a second end of the second element, the second end positioned adjacent to a milk drain.

10. A breast pump, comprising:
a breast cup for accommodating at least a portion of a user's breast;
a vacuum source connected to the breast cup, the vacuum source operatively connected to a controller, the controller programmed to provide a first baseline vacuum pressure to the breast cup and a second maximum vacuum pressure to the breast cup, at least a portion of the second maximum vacuum being at a vacuum pressure greater than the first baseline vacuum; and
a mechanical stimulator positioned within the breast cup, the mechanical stimulator including at least a first element and a second element, the first element moveable with respect to the second element.

11. The breast pump of claim 10, wherein the controller is programmed to operate said vacuum source in a manner that results in a vacuum waveform that cycles between the first baseline vacuum and the second maximum vacuum.

12. The breast pump of claim 10, wherein the first baseline vacuum pressure is at least 20 mmHg and the second maximum vacuum pressure is at least 40 mmHg.

13. The breast pump of claim 11, wherein the user can selectively adjust a frequency of the cycles between the first baseline vacuum and the second maximum vacuum.

14. The breast pump of claim 10, wherein the movement of the first element with respect to the second element comprises peristaltic axial movement from a first end of the first element to a second end of the first element, the second end of the first element adjacent to a milk drain.

15. A breast pump having an areola stimulation assembly, the breast pump comprising:
a breast cup;
an areola stimulation assembly disposed within the breast cup to simulate cycling mouthing, the areola stimulation assembly comprises an upper element, a lower element, and a mechanism that moves the lower element with respect to the upper element in a first movement with a first direction and a first frequency and a second movement with a second direction and a second frequency; and
a vacuum source connected to the breast cup to simulate cycling sucking, the vacuum source operatively connected to a controller, the controller programmed to provide a first baseline vacuum pressure to the breast cup and a second maximum vacuum pressure to the breast cup, at least a portion of the second maximum vacuum being different than the first baseline vacuum.

16. The breast pump of claim 15, wherein the second movement comprises peristaltic motion and the second direction comprises a horizontal direction, the peristaltic motion moves in a horizontal direction from an anterior position to a posterior position.

17. The breast pump of claim 15, wherein the first movement comprises opening and closing, and the first direction comprises a vertical direction, the opening of the first movement comprises a first spacing and the closing of the first movement comprises a second spacing, the second spacing being less than the first spacing.

18. The breast pump of claim 15, wherein the upper element comprises a flexible material, the upper element including a first end and a first width and a second end and a second width, the first end having a first width greater than the second width of the second end.

19. The breast pump of claim 16, wherein second frequency of the peristaltic motion of the second movement comprises at least 1 Hz or greater.

20. The breast pump of claim 17, wherein the first frequency of the opening and closing of the first movement comprises at least 1 Hz or greater.
